(19) United States
(12) Patent Application Publication
(10) Pub. No.: US 2011/0136162 A1
(43) Pub. Date: Jun. 9, 2011

(54) Compositions and Methods for Functionalized Patterning of Tissue Engineering Substrates Including Bioprinting Cell-Laden Constructs for Multicompartment Tissue Chambers

(75) Inventors: Wei Sun, Cherry Hill, NJ (US); Jessica Snyder, Atco, NJ (US); Robert Chang, Cherry Hill, NJ (US); Eda Yildirim, Sylvania, OH (US); Alexander Fridman, Philadelphia, PA (US); Halim Ayan, Murray, KY (US)

(73) Assignee: Drexel University

(21) Appl. No.: 12/872,992

(22) Filed: Aug. 31, 2010

Related U.S. Application Data
(60) Provisional application No. 61/238,481, filed on Aug. 31, 2009.
(60) Provisional application No. 61/258,917, filed on Nov. 6, 2009.

(51) Int. Cl. C12Q 1/02; C12M 1/00; C12M 1/34; C23C 16/50; C23C 16/52

(52) U.S. Cl. 435/29; 435/287.1; 118/723 R; 118/696; 427/2.1

(57) ABSTRACT

The present invention relates to microfluidic systems and methods for monitoring or detecting a change in a characteristic of an input substance. Specifically, the invention relates to a model for in vitro pharmacokinetic study and other pharmaceutical applications, as well as other uses including computing, sensing, filtration, detoxification, production of chemicals and biomolecules, testing cell/tissue behavior, toxicology, drug metabolism, drug screening, drug discovery, and implantation into a subject. The present invention also relates to systems and methods of a microplasm functionalized surface patterning of a substrate. The present invention represents an improvement over existing plasma systems used to modify the surface of a substrate, as the present invention creates surface patterning without the use of a mask, stamp or a chemical treatment.

### CROSS-REFERENCE TO RELATED APPLICATIONS

This application claims the benefit of priority of U.S. patent application Ser. No. 61/238,481, filed Aug. 31, 2009, and U.S. patent application Ser. No. 61/258,917, filed Nov. 6, 2009, the entire disclosures of which are incorporated by reference herein as if each is set forth herein in its entirety.

### STATEMENT REGARDING FEDERALLY SUPPORTED RESEARCH OR DEVELOPMENT

This invention was made with government support under Grant No. 09940-008 awarded by NASA USRA. The U.S. Government has certain rights in the invention.

### BACKGROUND OF THE INVENTION

Tissue engineering (TE) is an emerging field for tissue repair and regeneration compared to conventional techniques including autograft and allograft, through engineering functional implants created from living cells. TE is a highly interdisciplinary research area where material science, engineering and biology are blended to achieve tissue regeneration (Vacanti, 2007, Proc Am Philos Soc. 151:395-402). Efforts have been put in laboratories around the world to regenerate liver, skin, bone, vascular, etc tissues by applying tissue engineering approach (Khan, et al., 2008, Journal of Bone and Joint Surgery-American 90A:36-42; Chang et al., 2008, Tissue Engineering Part C-Methods 14:157-166; Mansbridge, 2008, Journal of Biomaterials Science-Polymer Edition 19:955-968; Nerem, 2003, Atherosclerosis Supplements 4:265-265). To generate any type of tissue in a laboratory environment, scientists need to mimic the cellular microenvironment by offering structural, chemical, physical and biological cues to the cells (Recknor et al., 2006, Biomaterials 27:4098-4108). Introduction of these cues to the cellular environment starts with manufacturing a supportive matrix called a scaffold.

Various scaffold manufacturing techniques have been developed and are reported in literature including solvent casting, fiber bonding, phase separation, and salt leaching technology (Liu et al., 2004, Annals of Biomedical Engineering 32:477-486). Nevertheless, these technologies are often far from meeting the requirements of tissue engineering scaffolds. For instance, the solvent casting technique is a relatively easy fabrication process, but the scaffolds have low mechanical properties. Furthermore, the phase separation technique is able to manufacture scaffolds with high porosity but these scaffolds have lack of interconnectivity. In general the aforementioned techniques do not offer controlled architecture, with optimum mechanical characteristics, such as porosity and interconnectivity, which are essential for tissue engineered scaffolds. In this framework, solid free-form fabrication techniques can be used to manufacture designed scaffolds with predefined architecture, chosen material and desired mechanical properties (Sun et al., 2002, Computer Methods and Programs in Biomedicine 67:85-103). The Fused Deposition Modeling (FDM) technique is able to manufacture scaffolds with internal porous architecture (Hutmacher et al., 2004, Trends in Biotechnology 22:354-362). However, the initial drawback of FDM is that it requires filament preparation which is highly time consuming process. In addition, during the manufacturing process the filament needs to be straight and one piece. The unexpected buckling and break in the filament cause the manufacture to stop. On the other hand, a new Precision Extrusion Deposition (PED) system is able to manufacture tissue engineering scaffolds with essential features such as having three-dimensional (3D) structure with uniform pore size distribution, being reproducible, and having internal interconnectivity (Wang et al., 2004, Rapid Prototyping Journal 10:42-49).

Choosing the proper material for the engineered scaffold is important, as is the manufacturing techniques. In FDM and PED systems, thermoplastic materials are used because of the nature of the processing systems. Polycaprolactone (PCL) is suitable candidate for PED because of its low melting temperature (58° C.-60° C.), its structural stability and its less sensitivity to environmental conditions such as temperature, moisture (Engelberg et al., 1991, Biomaterials 12:292-304). In addition, polycaprolactone is biocompatible and biodegradable and is approved by FDA for numerous medical and drug delivery devices. However, when polycaprolactone is used in tissue engineering its physicochemical properties needs to be treated for improved cellular functions. The surface property of a material, especially the degree of hydrophilicity, plays an important role in cell adhesion during the initial period of cell seeding (Yildirim et al., 2008, Plasma Processes and Polymers 5:58-66). The chemical inertness and low surface energy of polycaprolactone causing an inadequate interaction with the biological surfaces can be changed by various surface treatment processes such as chemical treatment, thin film deposition, blending, ion beam radiation and plasma treatment (Oyane et al., 2005, Journal of Biomedical Materials Research Part A 75A, 138-145; Safinia et al., 2005, Biomaterials 26:7537-7547; Yang et al., 2002, Biomaterials 23:2607-2614). Among them, plasma treatment is the most versatile technique, because during the surface treatment it is only changing the surface properties while preserving the bulk properties. In addition, low temperature plasma treatment can be carried out at near-ambient temperature, thereby minimizing the risk of damage to heat-sensitive materials (Vasilets et al., 2006, High Energy Chemistry 40:79-85).

The ability to align cells and proteins and to guide their functions by providing engineered and designed environments has been a strong interest for a wide range of diagnostic and therapeutic applications (Singhvi et al., 1994, Science 264:696-698; Williams, 2009, Biomaterials 30:5897-5909). In the living tissue environment, cells and proteins are surrounded by topographic and biochemical cues which assist them to attach, align and guide their cell-cell and cell-substrate interaction (Curtis et al., 1990, Critical Reviews in Biocompatibility 5:343-362; Anselme, 2000, Biomaterials 21:667-681). In nature, these cues are inherently within the native biological system (Chen et al., 1997, Science 276:1425-1428; Stevens et al., 2005, Science 310:1135-1138). However, most currently used biomaterials often lack adequate surface structural or biochemical cues without an additional surface functionalization. To alter the surface functionality, a variety of techniques have been developed, for example, conventional photolithography (Lu et al., 2001, Biomaterials 22:291-297; Michel et al., 2002, Langmuir 18:3281-3287; Britland et al., 1992, Experimental Cell Research 198:124-129; Ber et al., 2005, Biomaterials 26:1977-1986; Patrito et al., 2007, Langmuir 23:715-719; Yap et al., 2007, Biomaterials 28:2328-2338; Dewez et al., 1998, Biomaterials 19:1441-1445), soft lithography (Whitesides et al., 2001, Annual Review of Biomedical Engineering 3:335-373; Miller et al., 2006, Biotechnology and Bioengineering 93:1060-1068), microcontact printing (Jackman et al., 1995, Science 269:664-666; Offenhausser et al., 2007, Soft Matter 3:290-298), self-assembled monolayers (SAMs) (Ostuni et al., 2001, Langmuir 17:6336-6343; Staii et al., 2009, Biomaterials 30:3397-3404), direct writing (Odde et al., 1999, Trends in Biotechnology 17:385-389), and laser ablation (Li et al., 2003, IEEE Transactions on Nanobioscience 2:138-145). These enabling surface treatment techniques can provide additional structural, chemical, and/or biological cues that regulate cells morphologies as well as the subsequent cellular function (Bakeine et al., 2009, Microelectronic Engineering 86:1435-1438; Itomare et al., 2008, Journal of Applied Biomaterials & Biomechanics 6:132-143).

For the development of tissue regeneration technology, scientists have been trying to mimic the microenvironment of the cells to improve cellular responses including attachment, proliferation and expression of differentiated phenotypes on polymeric tissue scaffolds (Zhang et al., 2009, Biomaterials 30(25):4063-9; Hutmacher et al., 2001, Journal of Biomedical Materials Research 55:203-216; Zeltinger et al., 2001, Tissue Engineering 7:557-72). One challenge in scaffold guided tissue engineering is to design and manufacture scaffolds with required mechanical integrity and regulating cellular microenvironment to provide structural, biological, physical and chemical cues to cells. While proper scaffold manufacturing techniques can offer structural cues through intricate scaffold internal architectures to sustain the mechanical integrity of the cellular environment in vitro, the presence of biological, chemical and physical cues on the scaffolds is often not readily available for some synthesized biopolymer materials (Yildirim et al., 2007, NEBC Bioengineering Conference, IEEE 33rd Annual Northeast, 243-244; Shor et al., 2007, Biomaterials 28(35), 5291-5297; Yildirim et al., 2008, Plasma Processes and Polymers 5:397-397). The introduction of bioactive ligands, such as extracellular matrix (ECM) components, onto the manufactured scaffolds is one way of providing biological cues to the cells in vitro environment. Fibronectin (FN), the most common adhesive glycoprotein in ECM has been widely incorporated as bioactive ligands to the scaffold surface to improve the binding strength of cells (Keselowsky et al., 2007, Biomaterials 28:3626-3631). Studies using various cell lines have shown that the biological cues created through protein adsorption on a scaffold surface can guide a cell to select which cellular action to perform, such as attachment, migration, proliferation, apoptosis, or differentiation (Kennedy et al., 2006, Biomaterials 27:3817-3824; Wilson et al., 2005, Tissue Engineering 11:1-18; Silva et al., 2004, Materials Science & Engineering C-Biomimetic and Supramolecular Systems 24:637-641). Besides structural and biological cues, physical and chemical cues are other important factors that need to be considered during the biomimetic design of cellular environment (Yildirim et al., 2007, NEBC Bioengineering Conference, IEEE 33rd Annual Northeast, 243-244; Yildirim et al., 2008, Plasma Processes and Polymers 5:397-397). Plasma functionalization is one technique that is used to modify synthetic materials to introduce to the physical and chemical cues by creating, for example, micro scale roughness and/or forming chemical functional groups on the material. Using such a technique, a plasma source ignited mostly in a chamber from various gases to create a bombardment of a homogeneous mixture of charged particles (e.g., electrons, ions), neutral radicals and excited molecules as well as by UV radiation on the polymer surface (Yildirim et al., 2007, NEBC Bioengineering Conference, IEEE 33rd Annual Northeast, 243-244; Yildirim et al., 2008, Plasma Processes and Polymers 5:397-397). The benefits of plasma functionalization over other surface modification techniques include that it is homogenous, so even the surface of a 3D scaffold with complex geometries can be modified, and that it can create surface roughness and controllable chemical composition simultaneously without changing the bulk properties of the biosubstrate. These unique features of plasma modification make it functional in tissue engineering arena for enhanced cell-material interaction. Existing plasma functionalization techniques are used to functionalize the entire surface of a substrate, or when only a portion of the substrate is desire to be functionalized, require the use of a mask, a stamp or a chemical treatment.

In most of the aforementioned technologies, the surface functionalization is achieved by applying cell-adhesive and cell-repellant biomolecules to the surface using patterned masks, patterned stamps or with chemical treatment. Though effective in attracting cells on the patterned surfaces, the preparation of patterned mask and patterned stamps is often costly and requires long processing times and special clean room instrumentation (Hwang et al., 2009, Lab on a Chip 9:167-170; Falconnet et al., 2004, Advanced Functional Materials 14:749-756). In addition, the harsh chemical and solvent used in the process may also damage the patterned bio-organic layers (Khademhosseini et al., 2007, Biomedical Microdevices 9:149-157; Itoga et al., 2004, Biomaterials 25:2047-2053). Furthermore, the mask or stamps used do not provide precision control over the degree of surface functionalization, especially when using patterns having complex geometries (Ruiz et al., 2007, Soft Matter 3:168-177; Lee et al., 2003, Bulletin of the Korean Chemical Society 24:161-162). Due to these limitations, plasma-based surface treatment techniques have recently been examined for in both structural and chemical functionalization for eliciting biological responses (Bretagnol et al., 2007, Sensors and Actuators B-Chemical 123:283-292; Cheng et al., 2009, Biomaterials 30:4203-4210; Beaulleu et al., 2009, Langmuir 25:7169-7176; Cui et al., 2008, Journal of Photopolymer Science and Technology 21:231-244; Mona et al., 2002, Plasmas and Polymers 7:89-101; Frimat et al., 2009, Analytical and Bioanalytical Chemistry 395:601-609).

Existing plasma functionalization techniques do not allow for functionalized patterning on the surface of a substrate material without the use of mask, stamps or chemical treatment. Thus, there exists a need in the art for improved systems and methods for functionalized patterning on the surface of a substrate material without the use of mask, stamps or chemical treatment. The present invention fulfills this need.

Further, the current use of chemical coatings and modifications for cell/matrix attachment of microfluidic channels leads to residue formation and subsequent channel occlusions. Published biological data show that existing in vitro microfluidic devices do not demonstrate good cell viability or preservation of normal in vivo cell-specific physiological function necessary to accurately perform pharmacokinetic studies on a long-term basis.

For example, U.S. Pat. No. 5,612,188 (Shuler et al.) discloses a multi-chamber, in vitro system to simulate an interconnected organ system under a processor control. The system allows for gas exchange and fluid circulation. Within each chamber, cells of various types can be cultured which are representative of a desired organ. The multi-compartmental cell culture system uses large components such as culture chambers, sensors, and pumps, which require the use of large quantities of culture media, cells and test compounds. This system is very expensive to operate and requires a large amount of space in which to operate. Because this system is on such a large scale, the physiological characteristics vary considerably from those found in an in vivo situation. It is impossible to accurately generate physiologically realistic conditions at such a large scale. U.S. Pat. No. 6,916,640 (Yu et al.) describes culturing cells in a bioreactor using multi-layered microencapsulated cells.

U.S. Pat. No. 6,197,575 (Griffith et al.) describes a system for culturing cells using controlled channel structures to induce desired cell behavior and a sensing system to detect cellular or other material responses such as changes in metabolic products. One disadvantage of this system is that it relies upon cell migration for cell seeding, with no possibility for direct positional control of cell placement.

U.S. Pat. No. 6,133,030 (Bhatia et al.) describes a method of positioning cells in patterns by surface modification of the substrate to promote cell-specific adhesion, followed by co-culturing a layer of cells on top of the cell-patterned layer. This might improve cell metabolic activity through more natural cell-cell interactions. However, this method is a 2-D cell patterning of the feeder layer and does not have the ability for 3-D positional control and patterning of cells.

U.S. Patent Application No. 2007/0037275 to (Shuler et al.) discloses a microscale cell culture device which comprises a fluidic network of channels segregated into discrete but interconnected chambers. The specific chamber geometry is designed to provide cellular interactions, liquid flow, and liquid residence characteristics that correlate with those found for the corresponding cells, tissues, or organs in vivo. The fluidics are designed to accurately represent primary elements of the circulatory or lymphatic systems. In one embodiment, these components are integrated into a chip format. The design and validation of these geometries is based on a physiological-based pharmacokinetic model, a mathematical model that represents the body as interconnected compartments representing different tissues. The device can be seeded with the appropriate cells for each culture chamber. For example, a chamber designed to provide liver pharmacokinetic characteristics is seeded with hepatocytes, and may be in fluid connection with adipocytes seeded in a chamber designed to provide fat tissue pharmacokinetics. The result is a pharmacokinetic-based cell culture system that represents the tissue size ratio, tissue to blood volume ratio, and drug residence time of the animal it is modeling. This reference does not describe creating an artificial three dimensional tissue incorporated into a microfluidic device and therefore, it is limited to interactions of cells seeded on the surfaces of the chamber.

U.S. Patent Application No. 2004/0259177 (Lowery et al.) described a high throughput screening system comprising a microfluidic device and a three-dimensional multicellular surrogate tissue assembly, wherein the cells are seeded within channels that mimic laminar flow through naturally occurring tissue.

U.S. patent application Ser. No. 12/297,305 describes a microfluidic system for monitoring or detecting a change in a characteristic of an input substance, but does not disclose the use of basement membrane extracts.

Therefore, despite the ongoing development, there is also a need for a more efficient microfluidic system employing basement membrane matrix (BME) for monitoring or detecting a change in a characteristic of an input substance in pharmacokinetic studies, as well as in other applications. The present invention also fulfills this need.

### BRIEF SUMMARY OF THE INVENTION

The present invention relates to a microfluidic system for monitoring or detecting a change in a characteristic of an input substance. The microfluidic system includes a cover platform having an inlet for delivery of an input substance and an outlet for removal of an output substance, a substrate platform having a tissue chamber in a substrate body of the substrate platform and a three-dimensional tissue analog comprising cells mixed with a basement membrane matrix (BME), a first microfluidic channel in fluid communication with the inlet for delivery of the input substance and the tissue chamber and a second microfluidic channel in fluid communication with the outlet for removal of the output substance, provided that the substrate platform and the cover platform are superimposed to form a sealed assembly, an input substance unit, and optionally a pumping assembly and a detecting unit.

In one embodiment, the substrate platform comprises the first microfluidic channel and the second microfluidic channel in fluid communication with the tissue chamber. In another embodiment, the cover platform comprises the first microfluidic channel and the second microfluidic channel in fluid communication with the tissue chamber. In another embodiment, at least one of the cover platform and the substrate platform comprises a surface with an improved hydrophilicity. In another embodiment, at least one of the cover platform and the substrate platform are made of a polymer, glass, a ceramic, a metal, an alloy, or a combination thereof. In another embodiment, the cover platform is made of a plasma treated glass and the substrate platform is made of a plasma treated biologically compatible polymer composed of a plurality of siloxane units. In another embodiment, the tissue analog is at least one selected from the group consisting of heart, stomach, kidney, intestine, lung, liver, fat, bone, cartilage, skeletal muscle, smooth muscle, cardiac muscle, bone marrow, muscle, brain, and pancreas. In another embodiment, the microfluidic system includes a plurality of microfluidic channels. In another embodiment, the microfluidic system includes a plurality of tissue chambers.

The present invention also relates to a method of monitoring or detecting a change in a characteristic of an input substance. The method includes the steps of providing the aforementioned microfluidic system, providing the input substance unit comprising the input substance, directing the input substance into the microfluidic system, wherein the input substance flows through the inlet for delivery of the input substance and the first microfluidic channel into the tissue chamber having the tissue analog, removing the output substance from the microfluidic system via the second microfluidic channel and the outlet for removal of the output substance, obtaining at least a portion of the input substance prior to entry into the microfluidic system and at least a portion of the output substance after exiting the microfluidic system, measuring the characteristic of the input substance prior to entry into the microfluidic system and measuring the characteristic of the output substance after exiting the microfluidic system, comparing the measured characteristic of the input substance prior to entry into the microfluidic system with the measured characteristic of the output substance after exiting the microfluidic system, and thereby monitoring or detecting a change in the characteristic of the input substance.

In one embodiment, the input comprises a drug. In another embodiment, monitoring or detecting the change in the characteristic of the input substance comprises collecting the output comprising a metabolite having a detectable characteristic, detecting the detectable characteristic, and correlating the detectable characteristic to at least the extent and rate of metabolism of the input substance.

The present invention also relates to a microplasma system for functionalized patterning of a tissue engineering substrate. The system includes a microplasma nozzle fixed adjacent to a substrate material that is affixed to a platform moveable by a motion control system to position and move the platform in the X, Y and Z directions in relation to the fixed microplasma nozzle to create a functionalized pattern on the surface of the substrate material. In one embodiment, the substrate material is polycaprolactone.

The present invention also relates to a microplasma system for functionalized patterning of a tissue engineering substrate. The system includes a moveable microplasm nozzle affixed to motion control system to position and move the microplasma nozzle in the X, Y and Z directions in relation to a substrate material to create a functionalized pattern on the surface of the substrate material.

In one embodiment, the microplasm nozzle is affixed to a multi-nozzle bioprinting system comprising a data processing system that processes a designed scaffold model and converts it into a layered process tool path, a motion control system driven by the layered process tool path, and a material delivery system comprising multiple nozzles of different types, wherein at least one of the nozzles deposits at least one substrate material, and at least one of the nozzles deposits at least one type of cell, and at least one of the nozzles deposits at least one biomolecule, thereby constructing a scaffold having a microplasma functionalized pattern.

The present invention also relates to a method of creating a functionalized pattern on the surface of a tissue engineering substrate. The method includes the steps of fixing a microplasma nozzle adjacent to a substrate material that is affixed to a platform moveable by a motion control system, and moving the platform in the X, Y and Z directions in relation to the fixed microplasma nozzle to create a functionalized pattern on the surface of the substrate material.

The present invention also relates to a method of creating a functionalized pattern on the surface of a tissue engineering substrate. The method includes the steps of moving a microplasm nozzle affixed to motion control system in the X, Y and Z directions in relation to a substrate material to create a functionalized pattern on the surface of the substrate material. In one embodiment, the microplasma nozzle is integrated into a multi-nozzle bioprinting system. In another embodiment, the substrate material is polycaprolactone.

### BRIEF DESCRIPTION OF THE DRAWINGS

The following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings. In the drawings:

FIG. 1, comprising FIGS. 1A and 1B, depicts a scheme demonstrating an exemplary process of bioprinting a tissue-on-a-chip (FIG. 1A) and exemplary process of making the microfluidic system of the invention (FIG. 1B).

FIG. 2, comprising FIGS. 2A-2C, depicts a top view of the microfluidic system of the invention without the tissue analog in the tissue chamber (FIG. 2A) and side views demonstrating a step of making the tissue analog in a tissue chamber of the microfluidic system of the invention (FIGS. 2B and 2C).

FIG. 3 depicts a top view of the microfluidic system of the invention with the tissue analog in the tissue chamber.

FIG. 4 depicts a scheme demonstrating a method of monitoring or detecting a change in a characteristic of an input substance based on Fluorescent Microplate Reader analysis for determining a concentration of a drug and a metabolite.

FIG. 5, comprising FIGS. 5A-5C, depicts a scheme demonstrating an exemplary design pattern for the tissue analog (FIG. 5A), a scheme demonstrating a sandwich pattern for a tissue-on-a-chip application and a sample CAD model of a microfluidic chamber housing 3D microorgan (FIG. 5B), and a scheme demonstrating a sandwiched construct which simulates diffusion in all directions (FIG. 5C).

FIG. 6 depicts the results of an example experiment demonstrating hepatocyte cell viability as a function of process characteristics.

FIG. 7 depicts the results of an example experiment demonstrating hepatocyte urea synthesis of 3D cell-encapsulated alginate versus 2D static cell culture.

FIG. 8 depicts a sample schematic of a “lab on a chip.”

FIG. 9 depicts a sample schematic of an embodiment of a temperature-controlled basement membrane matrix (BME) printing system of the invention.

FIG. 10 depicts a picture of an embodiment of a temperature-controlled basement membrane matrix (BME) printing system of the invention.

FIG. 11 depicts the results of an example experiment evaluating the radiation sensitivity of cells treated with amifostine.

FIG. 12, comprising FIGS. 12A-12C, depicts the results of an example experiment evaluating the viability of the cells printed with the system. FIG. 12a: Unprinted samples counterstained with DNA stain Hoechst 33342 and cell-impermeant Alexa Fluor® 594 wheat germ agglutinin (WGA); FIG. 12b: Samples printed at 5 psi with 400 μm nozzle and counterstained with DNA stain Hoechst 33342 and cell-impermeant Alexa Fluor® 594 wheat germ agglutinin (WGA); FIG. 5c: Samples printed at 40 psi with 150 μm nozzle and counterstained with DNA stain Hoechst 33342 and cell-impermeant Alexa Fluor® 594 wheat germ agglutinin (WGA

FIG. 13 depicts a schematic of an embodiment of a microfluidic dual micro-organ.

FIG. 14 depicts a schematic of an embodiment of a microfluidic dual micro-organ.

FIG. 15 depicts a schematic XYZ positioner and material extrusion system in PED (a) and a schematic of a material extrusion system and its components (b).

FIG. 16 depicts the interface of an example system control software in PED.

FIG. 17 depicts an example of a designed scaffold used in the PED resolution test.

FIG. 18 depicts the top view of an example scaffold manufactured with PED.

FIG. 19 depicts the results of an example experiment assessing the polar and dispersive component of total surface energy (mN/m) for various plasma treatment time durations.

FIG. 20 depicts the results of an example experiment assessing the total surface energy (mN/m) for various plasma treatment time durations.

FIG. 21 depicts the results of an example experiment assessing the normalized fluorescence intensity for various plasma treatment times (0, 0.5, 1, 2, 3, 5 and 7 minutes) of plasma treated polycaprolactone after applying 27 dynes/cm² shear stress in the parallel-plate flow chamber.

FIG. 22 depicts the results of an example experiment assessing the fluorescence intensity of cells on plasma treated and untreated polycaprolactone scaffolds over 7 days. The error bars represent±standard deviation with n=4 for each group and each measurement day.

FIG. 23 depicts the single linear surface energy regression from the contact angle data of various probe liquids.

FIG. 24 depicts a schematic view of shear flow assay apparatus.

FIG. 25 depicts the results of an example experiment assessing the polar, dispersive and total surface energy (mN/m) of plasma, protein and plasma/protein modified polycaprolactone

FIG. 26 depicts Atomic Force Microscopy (AFM) phase images of polycaprolactone surface (a) Unmodified, (b) Protein coated, (c) Plasma modified, (d) Plasma/Protein modified.

FIG. 27 depicts the survey X-ray Photoelectron Spectroscopy (XPS) spectra of polycaprolactone surface (a) Unmodified, (b) Protein coated, (c) Plasma modified, and (d) Plasma/Protein modified.

FIG. 28 depicts the deconvoluted C₁ₛ XPS spectra of polycaprolactone surface (a) Unmodified, (b) Protein coated, (c) Plasma modified, and (d) Plasma/Protein modified.

FIG. 29 depicts the results of an example experiment assessing the cell number on unmodified and modified polycaprolactone after applying shear flow corresponding to 27 dynes/cm² shear stress in the parallel-plate flow chamber.

FIG. 30 depicts the results of an example experiment assessing the cell number on unmodified and modified polycaprolactone scaffolds over 21 days of culture in osteogenic medium. The error bars represent±standard deviation with n=4 for each group and each measurement day.

FIG. 31 depicts the results of an example experiment assessing the alkaline phosphatase activity (ALP) on unmodified and modified polycaprolactone scaffolds for 21 days of vitro culture. The results are expressed as means±standard deviation with n=4 for each group and each measurement day.

FIG. 32 depicts the results of an example experiment assessing the amount of osteocalcin protein secreted in the osteogenic medium by mouse osteoblast cells cultured on 3D polycaprolactone scaffolds up to 21 days. The results are expressed as means±standard deviation with n=4 for each group and each measurement day.

FIG. 33 depicts a schematic view of an embodiment of a microplasma system.

FIG. 34 depicts a schematic of an embodiment of an integrated microplasma printing system and its components.

FIG. 35 depicts the results of an example experiment assessing the effect of microplasma surface functionalization patterning on surface chemistry.

FIG. 36 depicts the results of an example experiment assessing cell morphology on unmodified (A) and microplasma modified (B) samples by scanning electron microscopy.

### DETAILED DESCRIPTION OF THE INVENTION

The present invention relates to microfluidic systems and methods for monitoring or detecting a change in a characteristic of an input substance. Specifically, the invention relates to a model for in vitro pharmacokinetic study and other pharmaceutical applications, as well as other uses including computing, sensing, filtration, detoxification, production of chemicals and biomolecules, testing cell/tissue behavior, toxicology, drug metabolism, drug screening, drug discovery, and implantation into a subject.

The present invention also relates to systems and methods of a microplasm functionalized surface patterning of a substrate. The present invention represents an improvement over existing plasma systems used to modify the surface of a substrate, as the present invention creates surface patterning without the use of a mask, stamp or a chemical treatment.

In some embodiments, the microplasm functionalized surface patterning of a substrate is used in conjunction with a cell printing system and method. When used in combination with a cell printing system, the microplasm systems and methods of the invention create patterned cells on various substrates without using a mask, a stamp or a chemical treatments.

In other embodiments, the microplasm functionalized surface patterning of a substrate is used in conjunction with biomolecule printing system and method. When used in combination with a cell printing system, the microplasm systems and methods of the invention create patterned biomolecules on various substrates without using a mask, a stamp or a chemical treatments.

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent based on the context in which it is used.

The term “tissue-on-a chip” as used herein means the microfluidic system of the invention wherein the tissue analog is bioprinted into a chamber located in the substrate platform, which is joined with a cover platform to form the microfluidic system in a shape of a chip.

The term “bioprinting” as used herein means a process of making a tissue analog by depositing scaffolding material (e.g., BME) alone, or mixed with cells, based on computer driven mimicking of a texture and a structure of a naturally occurring tissue.

A “stabilizing agent,” as used herein, is an agent used to stabilize drugs and provide a controlled release. Such agents include albumin, polyethyleneglycol, poly(ethylene-co-vinyl acetate), and poly(lactide-co-glycolide).

The term “attached,” as used herein encompasses interaction including, but not limited to, covalent bonding, ionic bonding, chemisorption, physisorption and combinations thereof.

The term “biomolecule” or “bioorganic molecule” refers to an organic molecule typically made by living organisms. This includes, for example, molecules comprising nucleotides, amino acids, sugars, fatty acids, steroids, nucleic acids, polypeptides, peptides, peptide fragments, carbohydrates, lipids, and combinations of these (e.g., glycoproteins, ribonucleoproteins, lipoproteins, or the like).

The term “differentiation factor,” as used herein, refers to a molecule that induces a stem cell or progenitor cell to commit to a particular specialized cell type.

“Extracellular matrix” or “matrix” refers to one or more substances that provide substantially the same conditions for supporting cell growth as provided by an extracellular matrix synthesized by feeder cells. The matrix may be provided on a substrate. Alternatively, the component(s) comprising the matrix may be provided in solution. Components of an extracellular matrix can include laminin, collagen and fibronectin.

A “growth environment” is an environment in which cells will proliferate in vitro. Features of the environment include the medium in which the cells are cultured, and a supporting structure (such as a substrate on a solid surface) if present.

“Growth factor” refers to a substance that is effective to promote the growth of cells. Growth factors include, but are not limited to, basic fibroblast growth factor (bFGF), acidic fibroblast growth factor (aFGF), epidermal growth factor (EGF), insulin-like growth factor-I (IGF-T), insulin-like growth factor-II (IGF-II), platelet-derived growth factor-AB (PDGF), vascular endothelial cell growth factor (VEGF), activin-A, bone morphogenic proteins (BMPs), insulin, cytokines, chemokines, morphogens, neutralizing antibodies, other proteins, and small molecules.

“Hydrogel” refers to a water-insoluble and water-swellable cross-linked polymer that is capable of absorbing at least 3 times, preferably at least 10 times, its own weight of a liquid. “Hydrogel” can also refer to a “thermo-responsive polymer” as used herein.

As used herein, “scaffold” refers to a structure, comprising a biocompatible material, that provides a surface suitable for adherence and proliferation of cells. A scaffold may further provide mechanical stability and support. A scaffold may be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

The term “isolated” refers to a material that is substantially or essentially free from components, which are used to produce the material. The lower end of the range of purity for the compositions is about 60%, about 70% or about 80% and the upper end of the range of purity is about 70%, about 80%, about 90% or more than about 90%.

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

As used herein, a “graft” refers to a cell, tissue or organ that is implanted into an individual, typically to replace, correct or otherwise overcome a defect. A graft may further comprise a scaffold. The tissue or organ may consist of cells that originate from the same individual; this graft is referred to herein by the following interchangeable terms: “autograft,” “autologous transplant,” “autologous implant” and “autologous graft”. A graft comprising cells from a genetically different individual of the same species is referred to herein by the following interchangeable terms: “allograft,” “allogeneic transplant,” “allogeneic implant” and “allogeneic graft”. A graft from an individual to his identical twin is referred to herein as an “isograft,” a “syngeneic transplant,” a “syngeneic implant” or a “syngeneic graft”. A “xenograft,” “xenogeneic transplant” or “xenogeneic implant” refers to a graft from one individual to another of a different species.

As used herein, the terms “tissue grafting” and “tissue reconstructing” both refer to implanting a graft into an individual to treat or alleviate a tissue defect, such as a lung defect or a soft tissue defect.

An “isolated cell” refers to a cell which has been separated from other components and/or cells which naturally accompany the isolated cell in a tissue or mammal.

As used herein, a “substantially purified” cell is a cell that is essentially free of other cell types. Thus, a substantially purified cell refers to a cell which has been purified from other cell types with which it is normally associated in its naturally-occurring state.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of 3H-thymidine into the cell, and the like.

As used herein, “tissue engineering” refers to the process of generating tissues ex vivo for use in tissue replacement or reconstruction. Tissue engineering is an example of “regenerative medicine,” which encompasses approaches to the repair or replacement of tissues and organs by incorporation of cells, gene or other biological building blocks, along with bioengineered materials and technologies.

In some embodiments, the systems and methods of the present invention can perform metabolic and cytotoxicity studies on a microscale that is comparable to human physiologic scales. In other embodiments, the compositions and methods of the invention can be utilized for drug screening methods having high-throughput capability and portability that can lead to significant cost reductions attributed to reduced time and effort in the number of animal and human trial studies conducted. A suitable in vitro drug screening processes can aid in new drug discovery processes.

The invention also includes methods of making a microfluidic system. The fabrication process of bioprinting, as described herein, has been developed to build a 3-dimensional heterogeneous cell-encapsulated BME-based construct within a microfluidic system which serves as a fluid circulator and as a platform for experimental drug/chemical analysis and toxicology.

The present invention includes an in vitro model that can be employed to predict an animal's response to various drug administrations and toxic chemical exposure. By fabricating a three-dimensional in vitro tissue analog comprising an incorporated array of microfluidic channels and tissue-embedded chambers, one can selectively biomimic different mammalian tissues for a multitude of applications. One such nonlimiting example is liver tissue for experimental pharmaceutical screening of drug efficacy and toxicity.

An example approach to the construction of such an in vitro model includes 1) the development of a viable bioprinting freeform fabrication process for making a bioprinted tissue by, for example, a layer-by-layer deposition of a three-dimensional cell-encapsulated BME-based tissue construct, and 2) the direct printing of the tissue construct onto a plasma surface-treated microfluidic system. Accordingly, in one embodiment, the invention is a microfluidic system for monitoring or detecting a change in a characteristic of an input substance which includes: (1) a microfluidic system, wherein the microfluidic system includes a microfluidic system, wherein the microfluidic system comprises (a) a cover platform having an inlet for delivery of an input substance and an outlet for removal of an output substance, (b) a substrate platform having (i) a tissue chamber in a substrate body of the substrate platform and (ii) a three-dimensional tissue analog comprising cells mixed with a BME, (c) a first microfluidic channel in fluid communication with the inlet for delivery of the input substance and the tissue chamber, and (d) a second microfluidic channel in fluid communication with the outlet for removal of the output substance, provided that the substrate platform and the cover platform are superimposed to form a sealed assembly; and optionally (2) a pumping assembly; and (3) a detecting unit.

As described herein, a tissue analog can be directly printed into a tissue chamber created using soft lithographic techniques (e.g., nanotransfer printing, microtransfer molding, replica molding, micromolding in capillaries, near field phase shift lithography, and solvent assisted micromolding; see, for example U.S. Pat. No. 7,195,733 to Rogers et al.) and used as a flow mimicking reservoir thus replacing the previously described microchannels seeded with cells. MEMS microfabrication can also be used for biochip fabrication and simulating microflow conditions. Cells are generally seeded after fabrication of the microfluidic system to grow within the microchannels. SFF can create complex 3-D shapes, and deposit biomaterials and cells for tissue engineering, but it is not as useful as MEMS microfabrication in incorporating complex electromechanical elements, actuators, and valves to create microflow systems. Advantageously, the inventors have combined the two processes to provide much greater benefit than either process by itself and overcomes the limitations of either method. SFF can be used to deposit/seed cells directly into channels or other positional locations within the microfluidic system and build tissue constructs within chambers that exhibit spatial patterning.

The bioprinting system described herein provides a biofriendly environment (e.g., no use of excessive pressure, heat or toxic chemicals) for single or multi-nozzle bioprinting capability for reproducibly making complex, three-dimensional, heterogeneous tissue analog constructs. SFF techniques useful in the compositions and methods of the invention include, but are not limited to, 3DP, syringe dispensing, piezoelectric glass capillary jetting, thermal and ink-jetting, solenoid valve-based jetting, polymer-based UV curing, deposition, and sprays.

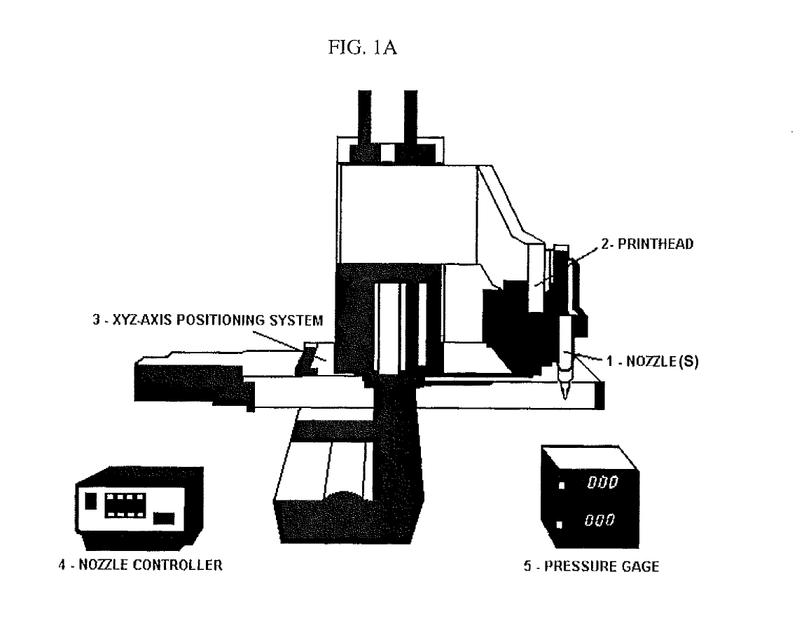

A single or multi-nozzle bioprinting system can be used in the methods of the invention described herein. An exemplary embodiment of a bioprinting system is illustrated in FIG. 1A (front view), it consists of one or more nozzles 1 mounted on a printhead 2. The printhead 2 is attached to a computer-controlled XYZ axis-positioning system 3. Dispensing of material is handled by the nozzle controller 4. Gages 5 are used to monitor process characteristics such as pressure.

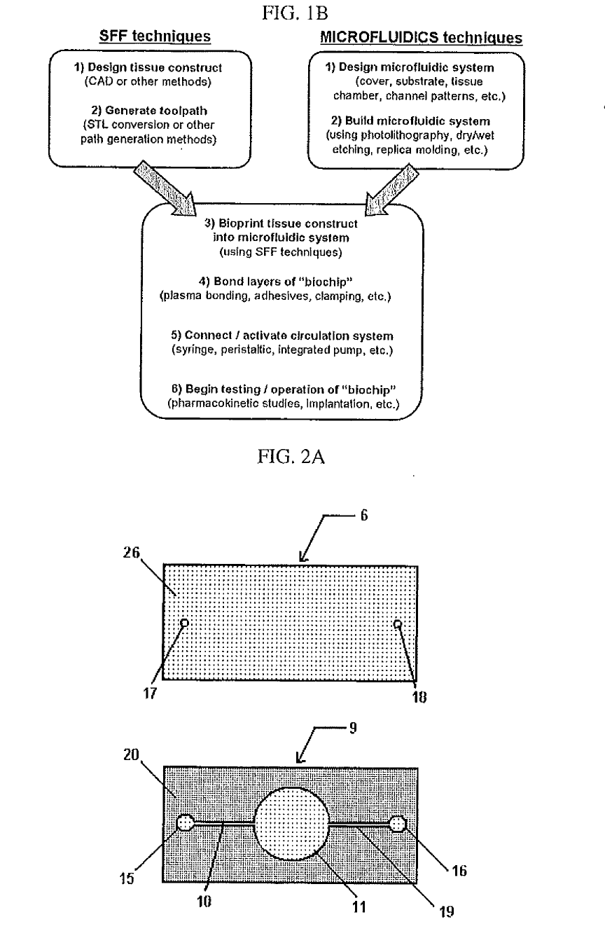

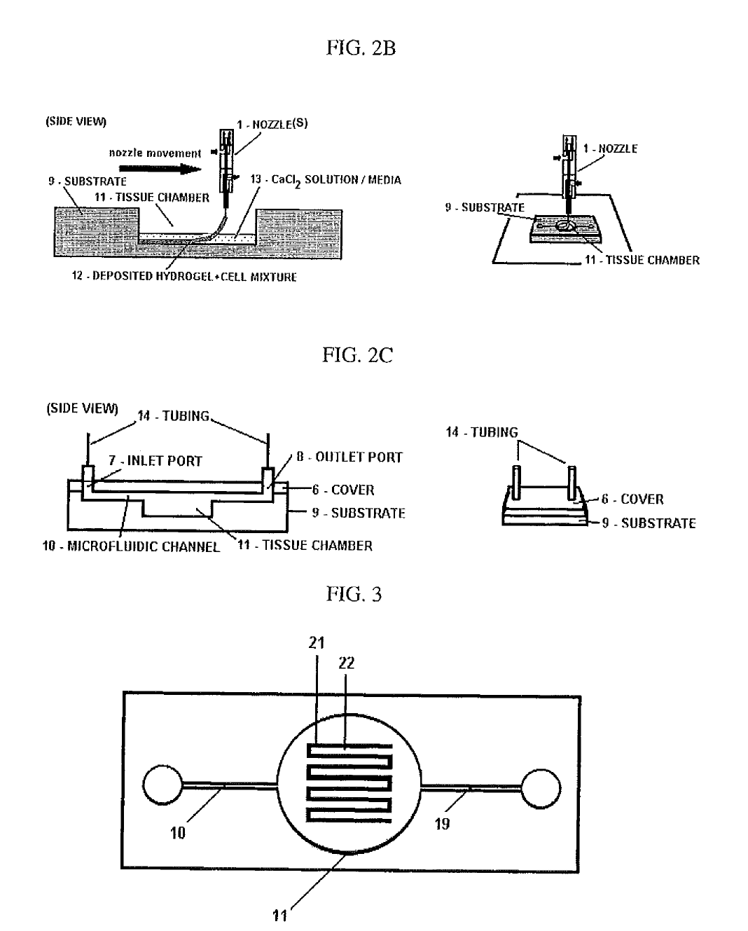

The bioprinting system is used to build 3-D tissue constructs within a microfluidic system (see FIG. 1B) as shown in FIGS. 2A-2C. FIG. 2A shows a basic, 2-platform embodiment of a microfluidic system of the microfluidic system of the invention.

FIG. 2A is a top view of the microfluidic system of the invention. It comprises two major units: a cover platform 6 and a substrate platform 9 which are superimposed and are held together by various ways, such as, for example an assembly of a screw and a nut. In a preferred embodiment, no additional means for holding the platforms are required; having been plasma treated, the two platforms can form a strong irreversible bond to prevent leaking.

The cover platform 6 comprises a cover body 26, an inlet port 7 and an outlet port 8 located on opposite sides of the cover platform 6, an inlet opening 17 (FIG. 2C) and an outlet opening 18 (FIG. 2C) attached to or integrated with corresponding inlet port 7 and an outlet port 8 positioned on opposite sides of the cover body 6 such that the inlet opening 17 and the outlet 25 opening 18 are positioned on the top portion of the cover body 6 and superimposed with the inlet port 7 and the outlet port 8; tubing 14 connected to the inlet opening 17 and the outlet opening 18 for delivery of an input medium and removal of an output medium. It should be understood that the inlet port and the outlet port can have different shapes which are not limited to a cylinder shape; the ports can also be integrated as a single unit with the corresponding opening as well as with corresponding tubing.

The cover body can be manufactured from glass or other suitable materials, a polymer, ceramic, metal, alloy, or any combination thereof. In a preferred embodiment, the cover body is made of glass. In various embodiments, the glass or other suitable materials are plasma treated to provide improved hydrophilicity. Methods of plasma treatment are known in the art, see, for example U.S. Pat. No. 6,967,101 (Larsson et al.) and U.S. Pat. No. 5,028,453 (Jeffrey et al.).

The substrate platform 9 comprises a substrate body 20, a tissue chamber 11, a microfluidic channel 10 for an input media (a first microfluidic channel) and a microfluidic channel 19 for an output media (a second microfluidic channel) wherein each microfluidic channel is connected with an input entry compartment 15 and an output removal compartment 16. The input entry compartment 15 and the output removal compartment 16 are indentations or depressions in the substrate body 20 which are designed to assure smooth flowing of both input and output substance delivered from the inlet port 7 and removed from the outlet port 8. The input entry compartment 15 and the output removal compartment 16 can be deeper and/or wider than the microfluidic channels they are connected to. The microfluidic channels are etched or otherwise indented conduits which provide a delivery route for an input medium to the tissue analog located in the tissue chamber 11 and a removal route for the output medium from the tissue analog.

In certain embodiments, the delivery route for an input medium and the removal route for the output medium can be modified such that the microfluidic channels are etched in the cover body or partially etched in the cover body and partially etched in the substrate body. It should be understood that the purpose of the microfluidic channels is to deliver and remove the medium to and from the tissue analog in a closed assembly of the cover platform and the substrate platform.

In various embodiments, the tissue chamber of the microfluidic system has various shapes, e.g., square, oval, irregular, etc. In certain embodiments, a square tissue chamber is etched in the substrate platform; microchannels are etched in the glass platform and direct flow into the tissue chamber on the bottom layer. The tissue chamber 11 is located approximately in the middle of the substrate body 20. More than one tissue chamber can be utilized in the same substrate body. In certain embodiments, multiple tissue chambers would have an independent set of input/output routes; in other embodiments, several tissue chambers can be placed consecutively one after another and utilize various input/output routes or a single input/output route.

The substrate can be manufactured from the following exemplary materials: a polymer, ceramic, glass, metal, alloy, or any combination thereof. In preferred embodiments, the polymer comprises a biologically-compatible polymer. Suitable biologically-compatible polymers include a plurality of units derived from a siloxane, an alkyl oxide such as ethylene oxide, an acrylic, an amide, a polymerizable carboxylic acid group, or any combination thereof When the biologically-compatible polymers include a plurality of units derived from a siloxane, the siloxane units typically include a plurality of monomers that include dimethyl siloxane, or any combination thereof. A preferred biologically-compatible polymer composed of a plurality of siloxane units is polydimethyl siloxane (“PDMS”). Any other type of polymeric material that can be fabricated into optically transparent microfluidic systems, for example polymethylmethacrylate (“PMMA”), can also be used. The substrate material has to meet the primary requirement of biocompatibility and hydrophilicity. It is preferred that the substrate materials are plasma treated to provide permanent bonding as well as improved hydrophilicity for the PDMS substrate.

The cover body and substrate body materials that are not necessarily biologically compatible can also be used in some embodiments of the present invention. In these embodiments, substrate materials that are not alone biologically-compatible can be made compatible using a suitable surface treatment or coating to make them biologically-compatible.

Suitable surface treatments or coatings can include a film of a biologically-compatible material applied to the surface of a typically biologically-incompatible substrate. For example, the microfluidic structures patterned in a biologically-incompatible substrate can be surface treated with an optional adhesion modifying agent and then coated with a thin film of a biologically-compatible material, such as PDMS. Making indentation or etchings in the substrate can be done by methods known in the art, for example dry etching techniques such as deep-reactive ion etching, wet etching techniques using acids, and replica molding techniques. PDMS base and curing agent can be poured into a mold, degassed under vacuum, and then heated to create the PDMS platform. Tissue chamber 11 is designed to serve as a compartment or a “mold” for a tissue analog 21 with a pattern of inner channels 22 which can mimic a pattern of naturally occurring vessels as shown in FIG. 3. FIG. 3 is a top view of the microfluidic system of the invention with the tissue analog in the tissue chamber. The tissue analog is deposited from a nozzle of a bioprinting system which is operated based on computerized calculations and allows mimicking a desired tissue as a three dimensional construct. An exemplary bioprinting system is described in PCT/US2004/015316 published as WO 2005/057436 and in U.S. Patent Application Publication 2006/0105011), all incorporated herein in its entirety.

In preferred embodiments, the bioprinting material is a BME, such as that derived from Engelbreth-Holm-Swarm (EHS) sarcoma cells, which when chilled to, for example, 1-9° C., becomes suitably liquid for deposition, and which when warmed to, for example, 25-40° C., self-assembles and becomes more solidified. Non-limiting examples of such BME includes Matrigel® (BD Biosciences, San Jose, Calif.) and Cultrex®.(Trevigen, Gaithersburg, Md.). Prior to, during or after printing, the BME is mixed with the chosen cell type or types known to be present in a particular tissue depending on the desired application. In another embodiment, the bioprinting material is a biopolymer, such as a hydrogel, for example, alginate, which is mixed with cells known to be present in a particular tissue or other cells depending on a desired application. In still another embodiment, the bioprinting material is a combination of BME and a biopolymer.

Thus, a three dimensional tissue analog is bioprinted directly in the tissue chamber 11. Depending on the design of the experiment for measuring and analyzing output, there may be an empty space left in the tissue chamber; preferably, the tissue chamber is filled entirely.

Upon completion of printing, the top and bottom layers are bonded together as shown in FIG. 2C. For this embodiment, cleaning of the two surfaces to be joined is done with 70% ethanol, acetone, and deionized water, then plasma treatment is used to bond the cover 6 to the substrate 9. For hydrophobic materials such as PDMS, plasma treatment can be done prior to bioprinting to improve surface hydrophilicity, wettability, and cell adhesion within the tissue chamber and microchannels.

The input substance is administered to the tissue analog 21 through the tubing 14, the inlet port 7, the inlet opening 17, the input entry compartment 15, and the microfluidic channel 10. The input substance can be administered with the help of a pump (not shown) or gravity forces. A pump (e.g., syringe pump, peristaltic pump, microfluidic pumps, etc.) can be used at a calculated flow rate for desired residence time or shear flow. The pressure created in the system of the invention should be monitored to ensure that the flow is achieved and the seal is not compromised or the tissue adversely affected.

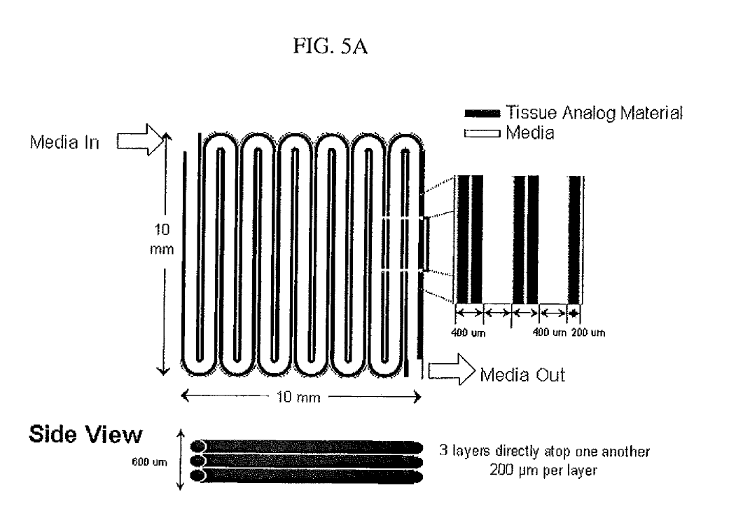

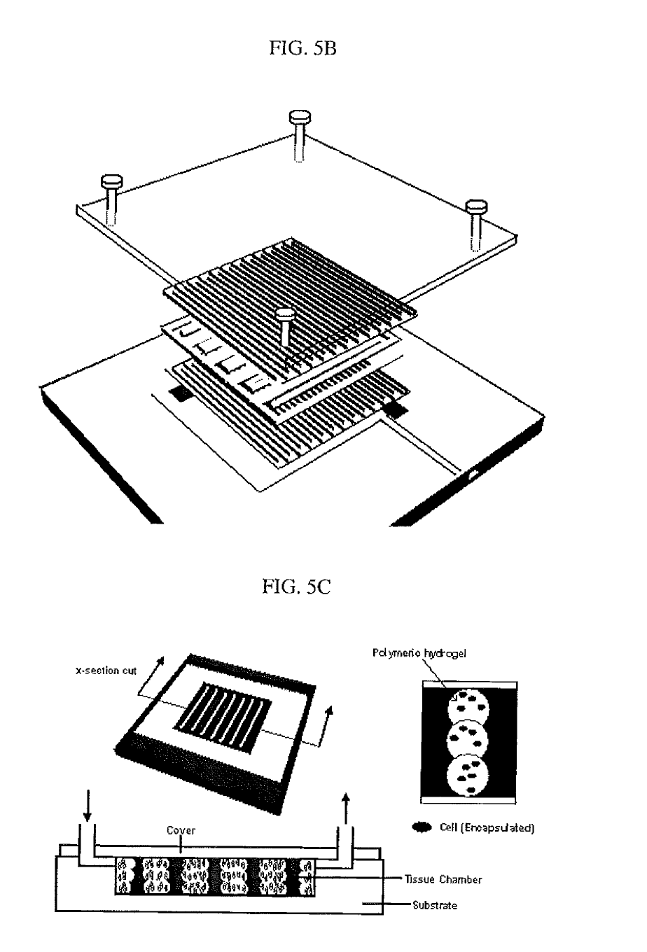

Once the input medium reached the tissue chamber 11 and the tissue analog 21, the input medium finds its way through the inner channels 22 (see FIG. 5A) and exits as an output into the microfluidic channel 19, the output removal compartment 16, the outlet opening 18, the outlet 20 port 8, and the tubing 14. The output is then collected and analyzed for a change in a selected characteristic of the tested material such as, for example, for metabolic activity or for reaction end products. Such analysis is conducted using methods well known in the art. Suitable assays involve measuring a change in a selected characteristic such as, for example, absorbance, fluorescence or nuclear magnetic resonance (NMR) properties of reporter molecules in a high throughput screening mode in 24, 48, or 96 well format currently used for drug candidate screening. It is envisioned that biochemical assay reporter molecules can be introduced into the microfluidic culture channels or produced by cells in the bioprinted tissue analog and direct measurements of change in the reporter molecule could be taken directly from the microfluidic system. This may provide a rapid method for verifying that compounds showing desired biochemical properties during initial screening and a corresponding inhibition or promotion of cell development are actually functioning as predicted. Further, a morphological analysis may be carried out using an inverted microscope; fluorescence labeling of cells, organelles, or macromolecules using exogenous fluors or expressed fluorescent proteins, such as green fluorescent protein, may be useful for detecting changes in cell properties. Enzyme linked immunosorbent assays (ELISA) may be used to determine the presence or quantity of, for example, growth factors. Metallo-proteases are often an indicator of tissue differentiation or tissue invasion and Zymogram gels (Invitrogen, Carlsbad, Calif.) are useful in measuring this activity. Micronuclei count can be used as an indicated of damage caused by radiation.

The embodiment depicted in FIG. 2B shows two different side views of the nozzle(s) 1 depositing a hydrogel mixed with a cell mixture 12 into a CaCl₂ crosslinking solution plus cell media 13 onto the substrate layer 9 within the tissue chamber 11. Complex patterns and structures can be created in this way through a layer-by-layer fashion. Finally, tubing 14 is connected to the inlet 7 and outlet 8 ports.

### Methods of Making Microfluidic Systems

Another aspect of the invention is a method of making the microfluidic system, which includes: fabricating the cover platform comprising a cover body, an inlet port, an inlet opening, an outlet port, an outlet opening, and optionally microfluidic channels using microfabrication techniques; fabricating the substrate platform comprising a substrate body, a tissue chamber, a first microfluidic channel and a second microfluidic channel wherein each microfluidic channel is in fluid communication with an input entry compartment and an output removal compartment, provided that each of the tissue chamber, the first microfluidic channel, the second microfluidic channel, the input entry compartment, and the output removal compartment represent indentations or depressions in the substrate body; plasma treating the substrate platform and the cover platform; making the tissue analog having the channel structure that can mimic a naturally occurring vessel network in the tissue analog three-dimensional construct comprising cells mixed with the tissue analog matrix by using a bioprinting freeform fabrication process for a layer-by-layer deposition of the tissue analog matrix comprising cells; forming the microfluidic system by superimposing the cover platform with the substrate platform such that the first microfluidic channel and the second microfluidic channel are in fluid communication with the tissue chamber, the an inlet port, the an outlet port, and the channel structure; and sealing the microfluidic system to provide the sealed assembly such that a flow of a substance can be conducted by engaging at least the inlet port, the first microfluidic channel, the second microfluidic channel, the channel structure, and the outlet port and thereby making the microfluidic system.

Microfabrication techniques such as photolithography, etching of silicon and glass, or replica molding and soft lithography techniques are well established in the literature, and can be used to create a wide variety of microfluidic systems.

As described elsewhere herein, experiments were done testing heterogeneous printing using a complex, multi-material part in CAD. For example, simultaneously deposited were materials containing different BME solutions admixed with cells and other biological factors. Three-dimensional hydrogel scaffolds have also been extruded as an alginate filament with the nozzle tip submerged within a crosslinking solution. The power of computer-aided design techniques is recruited to create hydrogel tissue constructs with various patterns. In order to ensure compatibility with a microscale cell culture analog system, boundary studies have been carried out with alginate testing the potential limits and capabilities of the bioprinting system, resulting in the creation of filaments within the 30-40 micron diameter range.

Preferably, the bioprinting materials of the invention should be biocompatible and biodegradable. In preferred embodiments, the bioprinting materials of the present invention is BME, alone or in combination with another material, such as a hydrogel. Hydrogels are useful biomaterials for 3D cell culture because of their high water content and mechanical properties resemble those of tissues in the body. One candidate hydrogel polymer that has demonstrated good cell viability and cell-specific function with the bioprinting process is sodium alginate, a co-block polysaccharide natural biopolymer.

In certain embodiments, a micro-scale tissue analog of the invention (e.g., a liver or other desired tissue) is fabricated via direct deposition of a three dimensional heterogeneous cell-seeded BME-based matrix. By integrating the bioprinting system with a CAD environment, notable feasibility and reproducibility of 3D structures within micron-order dimensional specifications have been realized. Repeated testing has demonstrated good cell viability and maintenance of liver cell-specific function for post-assembly bioprinted encapsulated hepatocytes (liver cells) under biofriendly conditions.

### Methods of Monitoring or Detecting a Change in a Characteristic of an Input Substance

In another embodiment, the invention includes a method of monitoring or detecting a change in a characteristic of an input substance which includes: providing a microfluidic system of the invention as described herein; providing the input substance unit comprising the input substance; directing the input substance into the microfluidic system, wherein the input substance flows through the inlet for delivery of the input substance and the first microfluidic channel into the channel network in the tissue analog; removing the input substance from the microfluidic system via the second microfluidic channel and the outlet for removal of the output substance; and obtaining at least a portion of the input substance prior to entry into the channel network and at least a portion of the output substance after exiting the channel network and thereby monitoring or detecting a change in the characteristic of the input substance.

### Pharmacokinetic Studies

Since hepatocytes are the cells that steward the metabolic and biosynthetic processes in the body, bioprinted liver tissue constructs are an exemplary chamber/compartment in microfluidic circuits. By combining SFF with microfluidics, an in vitro circulating system of drug perfusate is constructed for liver tissue analog construct functional analysis. Liver tissue is used herein as an example and should not be interpreted as a limitation to the invention as any tissue analog can be used in this invention.

Existing kinetic and thermodynamic equations may be written for each tissue construct/organ analog that describe the behavior of a drug or chemical in that organ. For example, in the liver compartment of a tissue-on-a-chip microsystem, a model drug compound is in large part metabolized by the cytochrome P450 monooxygenase system (CYP450) into reactive metabolites. Notably, clearance is an important characteristic in pharmacokinetics and provides a suitable basis for quantitative evaluation and comparison of fabricated liver tissue constructs with that of a normal human liver. The clearance of a drug is the volume of body fluid inflow from which the drug is completely removed by biotransformation and/or excretion, per unit time. Clearance is a pharmacokinetic characteristic which is experimentally evaluated as a function of varying design characteristics and biomaterial properties and subsequently optimized.

Rₘ=CLC₁

V₁ dC₁/dt=−QC₁+QC₂+R

V₂ dC₂/dt=−QC₂+QC₁+CL C₁
CL: volume of the inflow to the tissue analog from which the drug would be entirely removed in unit time
Rₘ: rate of metabolism in tissue analog
Q: circulating rate of perfusate
C₁: drug concentration entering tissue analog
C₂: drug concentration exiting tissue analog
R: constant rate of continuous infusion
D: Total amount of Drug in the medium

Initial Conditions:

C_{1|t=0}=D/V₁, C_{2|t=0}=0, R=0

CL is then obtained from the following relation:

A/α+B/β=D/CL

CL is dependent on

CL=Daβ/(βA+αB)

D—amount of drug which in turn relates to C₁

α, β—slope of graph which is dependent on cell density and cell type, biomaterial properties

A,B—intercept values of graph which is dependent on
Q=Flow rate of perfusate (medium+drug)
V2=Flow Volume of Construct Channel (Length×Cross Sectional Area)

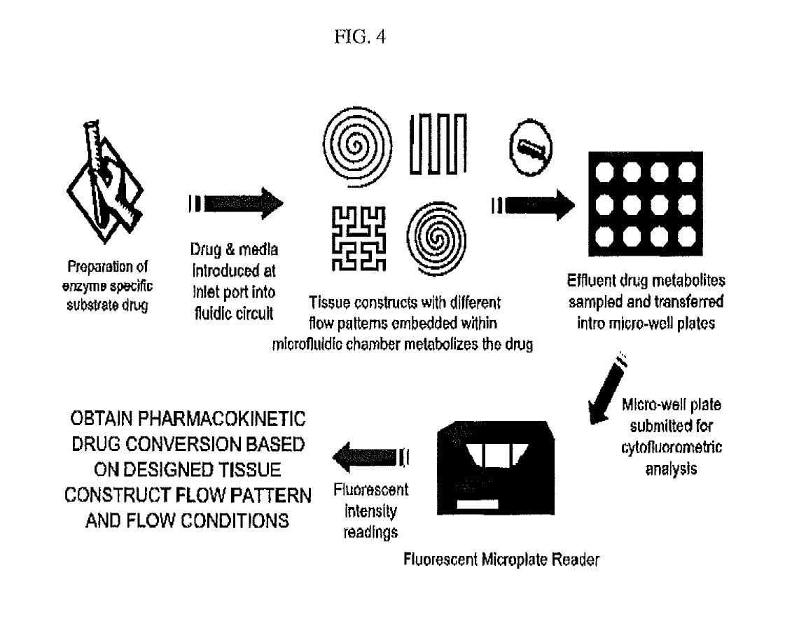

One way to demonstrate effective drug metabolism in the model system is to feed into the system a non-fluorescent prodrug that is metabolized by the liver tissue analog into a fluorescent metabolite that can be analyzed for relative fluorescent intensity, which is proportional to the relative drug metabolite concentration (FIG. 4). Such an analysis will provide information regarding the relative pharmacokinetic efficiency and relevancy of the microfabricated tissue-on a-chip of the invention for human application.

FIG. 4 shows a scheme demonstrating a process of Fluorescent Microplate Reader analysis for determining a concentration of a drug and a metabolite, wherein a mixture of a drug and a media is introduced at an inlet port into a fluidic circuit of a tissue construct of the invention with has a flow pattern of channels embedded within a microfluidic chamber. It should be understood that a flow pattern of channels can vary and is not limited to the patterns depicted in FIG. 4. Effluent drug metabolites are collected on micro-well plates to be tested, for example, with a Fluorescent Microplate Reader in accordance with known techniques.

### Three-Dimensional Tissue Analog Design

In certain embodiments, the microfluidic system of the invention is created using microfabrication techniques. For example, to create a polydimethosiloxane (PDMS) microfluidic system, a mold with microfluidic channels and a tissue chamber can be fabricated using photolithography with a negative photoresist such as SU-8. PDMS base and curing agent can be poured into the mold, degassed under vacuum, and then heated to create the PDMS layers or a platform.

The PDMS substrate is surface modified using, for example, air plasma treatment, to facilitate direct bioprinting. The substrate is placed within an RF plasma cleaner with vacuum applied for a minute to evacuate the chamber. The PDMS substrate is then exposed to the RF plasma for 30 seconds to improve surface hydrophilicity and adhesion properties to glass and surface treated PDMS.

BME-encapsulated cells are then printed into the tissue chamber of the plasma-treated substrate using SFF techniques in accordance with computer driven structure of a desired tissue analog. The model for the desired tissue structure is created on a computer and converted into a readable format for the XYZ-axis motion control system. Process characteristics such as printhead speed, pressure, nozzle inner diameter, temperature, and solution viscosity can be set depending upon the desired properties of the tissue construct.

Upon completion of the printing process, the two platforms are joined (e.g., adhered, bonded, or otherwise connected) together. Having been plasma treated, the two layers can form a strong irreversible bond to prevent leaking. The sealed microfluidic system containing the 3-D tissue analog is then connected to a syringe pump for controlled simultaneous infusion of a testing substance (e.g., a drug in an appropriate medium) at the inlet port and withdrawal at the outlet port.

In various embodiments, the pattern of the tissue analog varies. As depicted in

FIG. 5, a construct pattern is sandwiched in between at least two construct beds. The construct pattern can be created in a CAD environment (in silico), converted to an STL file and then converted into a toolpath. This toolpath can be used by the motion control software to direct the printhead and create the desired part. Alternatively, for a simple design, the toolpath can be created directly by using the motion control programming software. The ability to vary the geometry within the tissue chamber is one of the main advantages to combining SFF with microfluidics. The pattern can be as simple or as complex as desired. A standard biochip can be mass produced and can be tailored to many different functions by simply printing different constructs/patterns/cell types within the tissue chamber.

In FIG. 5B, the three layers represent the layered bioprinting fabrication approach to produce 3D tissue constructs within the chamber. Depending on the flow pattern specifications, the process toolpath leads to different patterns for each layer as well as orientation of each subsequent layer with respect to the preceding layer.

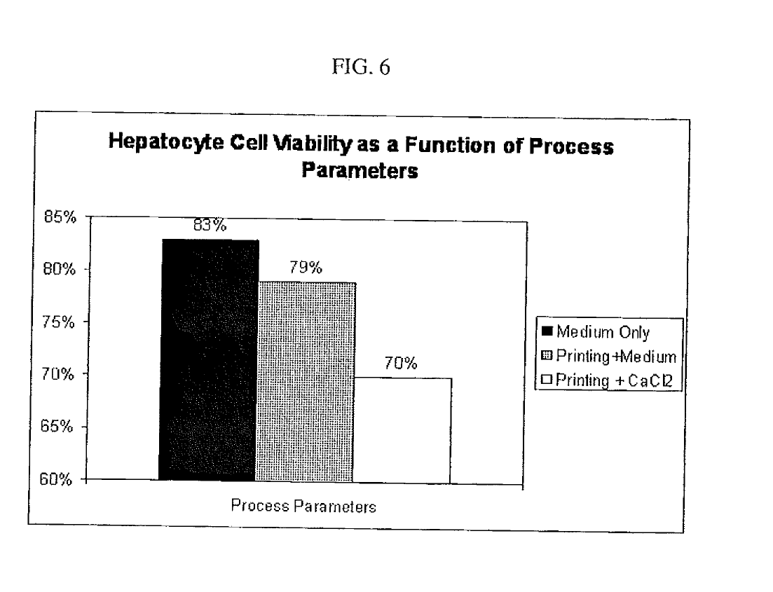

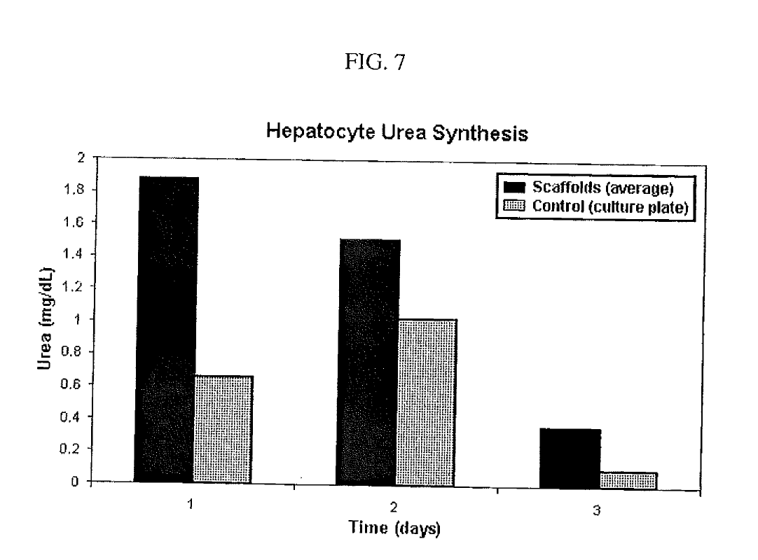

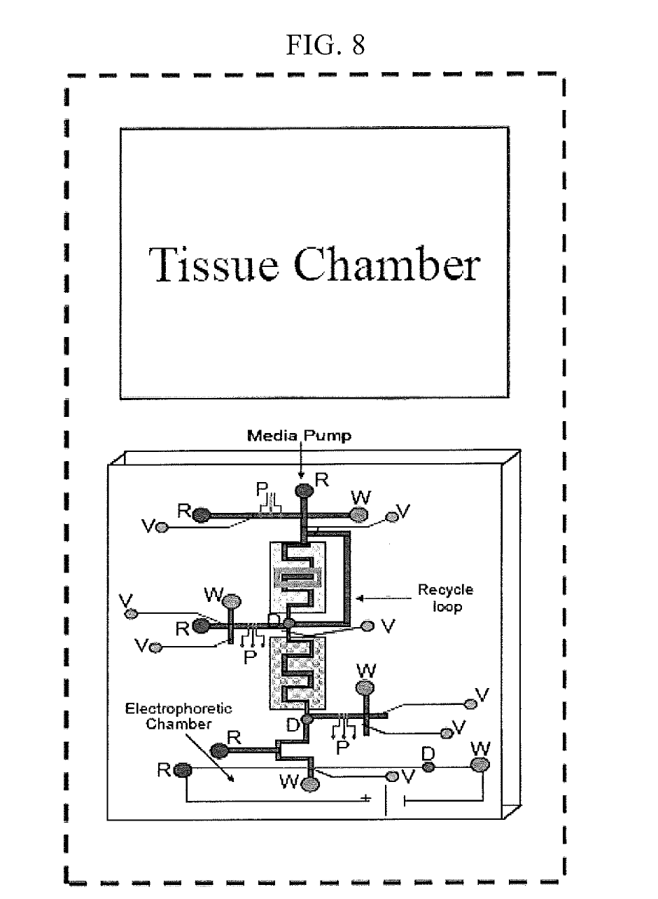

As shown in FIGS. 6 and 7, preliminary cell viability tests of the pneumatic printing process demonstrate that hepatocytes encapsulated in alginate were able to survive with a 79% cell viability ratio. The hepatocytes encapsulated in alginate synthesized a higher amount of urea than the same number of hepatocytes cultured on tissue culture plastic.

Other embodiments of the system/process could substitute different materials for the substrate such polymers, rubbers, plastics, metals, etc. depending upon the desired mechanical, electrical, biological, or other properties such as material strength, conductivity, cell adhesion, biocompatibility, or optical transparency/opacity.

In certain embodiments, glass layers can be used. Also chrome layers can be deposited as a mask with photolithography used to expose the desired channel pattern to be used as masks, or metal layers to be used as electrodes. Wet etching with fluoridic acid (HF) would create channel structures in the glass. Such techniques could be combined with abrasive operations using diamond-coated bits. For silicon layers, MEMS techniques such as deep RIB, wet etching, and other standard industry techniques can be used to create the desired geometry.

In other embodiments, the substrate platform can be modified in different ways such as plasma treatment, alterations in surface charge, covalent bonding of proteins and other moieties, surface roughening, oxidation, etching, and other surface modification procedures to create the desired properties, e.g., better cell adhesion, wetting properties, etc. Alternative embodiments could vary the bonding method of the layers such as chemical modification or the use of adhesives. Additionally, many multiple layers could be bonded together to form stacks of biochips.

Alternative embodiments can deliver the cells using non-gels such as liquid media, solids, or gases, and does not necessarily require cell encapsulation. Additional embodiments may not even use cells but deposit acellular material. For example, micelles, plasma membrane analogues, or other non-living components could be deposited for pharmacokinetic or other studies.

Other embodiments of the microfluidic system further include incorporating electrodes for directed electroosmotic and electrokinetic flow, or for heating, temperature regulation, and sensor functions, and also the incorporation of microvalves and micropumps known in the literature (Madou, 2002, Fundamentals of Microfabrication, CRC Press: New York; Tabeling, 2005, Introduction to Microfluidics, Oxford University Press: New York).

In some embodiments, the system includes a mechanism for obtaining signals from the cells of the tissue analog and/or the medium. The signals from different chambers and channels can be monitored in real time. For example, biosensors can be integrated or external to the system, which permit real-time readout of the physiological status of the cells in the system.

Any cell type is suitable for use with the invention described herein, such as for example, primary cells, stem cells, progenitor cells, normal, genetically-modified, genetically altered, immortalized, and transformed cell lines, single cell types or cell lines, or with combinations of different cell types. Preferably, the cultured cells maintain the ability to respond to stimuli that elicit a response in their naturally occurring counterparts. These may be derived from all sources such as eukaryotic or prokaryotic cells. The eukaryotic cells can be plant or animal, such as human, simian, or rodent. Cells useful in the invention can be of any tissue type (e.g., heart, stomach, kidney, intestine, lung, liver, fat, bone, cartilage, skeletal muscle, smooth muscle, cardiac muscle, bone marrow, muscle, brain, pancreas), and cell type (e.g., epithelial, endothelial, mesenchymal, adipocyte, and hematopoietic).

In addition, cells that have been genetically altered or modified so as to contain a nonnative “recombinant” (also called “exogenous”) nucleic acid sequence, or modified by antisense technology to provide a gain or loss of genetic function may be utilized with the invention. Methods for generating genetically modified cells are known in the art, see for example “Current Protocols in Molecular Biology,” Ausubel et al., eds, John Wiley & Sons, New York, N.Y., 2009. The cells could be terminally differentiated or undifferentiated, such as a stem cell. The cells of the present invention could be cultured cells from a variety of genetically diverse individuals who may respond differently to biologic and pharmacologic agents. Genetic diversity can have indirect and direct effects on disease susceptibility. In a direct case, even a single nucleotide change, resulting in a single nucleotide polymorphism (SNP), can alter the amino acid sequence of a protein and directly contribute to disease or disease susceptibility. For example, certain APO-lipoprotein E genotypes have been associated with onset and progression of Alzheimer's disease in some individuals.

### Input Variables

Drugs, toxins, cells, pathogens, samples, etc., herein referred to generically as “input variables” are screened for biological activity by adding them to the pharmacokinetic-based culture system, and then assessing the cultured cells or medium for changes in output variables of interest, e.g., consumption of O₂, production of CO₂, cell viability, metabolites, or expression of proteins of interest. The input variables are typically added in solution, or readily soluble form, to the medium of cells in culture. The input variables may be added using a flow through system, or alternatively, adding a bolus to an otherwise static solution. In a flow-through system, two fluids are used, where one is a physiologically neutral solution, and the other is the same solution with the test compound added. The first fluid is passed over the cells, followed by the second. In a single solution method, a bolus of the test input variables is added to the volume of medium surrounding the cells. In preferred embodiments, the overall composition of the culture medium should not change substantially with the addition of the bolus, or between the two solutions in a flow through method.

Preferred input variable formulations do not include additional components, such as preservatives, that have a significant effect on the overall formulation. Thus, preferred formulations include a biologically active agent and a physiologically acceptable carrier, e.g., water, ethanol, or DMSO. However, if an agent is liquid without an excipient, the formulation may be only the compound itself.

Preferred input variables include, but are not limited to, viruses, viral particles, liposomes, nanoparticles, biodegradable polymers, radiolabeled particles, radiolabeled biomolecules, toxin-30 conjugated particles, toxin-conjugated biomolecules, drugs, prodrugs, precursors, and particles or biomolecules conjugated with stabilizing agents.

A plurality of assays may be run in parallel with different input variable concentrations to obtain a differential response to the various concentrations. As known in the art, determining the effective concentration of an agent typically uses a range of concentrations resulting from 1:10, or other log scale, dilutions. The concentrations can be further refined with a second series of dilutions, if necessary. Typically, one of these concentrations serves as a negative control, i.e., at zero concentration or below the level of detection.

Input variables of interest encompass numerous chemical classes, though frequently they are organic molecules. A preferred embodiment is the use of the methods of the invention to screen samples for toxicity, e.g., environmental samples or drugs. Candidate agents may comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Included are pharmacologically active drugs and genetically active molecules. Non-limiting examples of compounds of interest include chemotherapeutic agents, anti-inflammatory agents, hormones or hormone antagonists, ion channel modifiers, anti-radiation drugs, and neuroactive agents. Exemplary of pharmaceutical agents suitable for this invention are those described in “The Pharmacological Basis of Therapeutics,” Goodman and Gilman, McGraw-Hill, New York, N.Y., (2006), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming Organs; Hormones and Hormone Antagonists; Vitamins, Dermatology; and Toxicology, all incorporated-herein by reference. Also included are toxins, and biological and chemical warfare agents, for example see Somani, S. M. (Ed.), “Chemical Warfare Agents,” Academic Press, New York, 1992).

Test compounds include all of the classes of molecules described above, and may further comprise samples of unknown content. While many samples will comprise compounds in solution, solid samples that can be dissolved in a suitable solvent may also be assayed. Samples of interest include environmental samples, e.g., ground water, sea water, or mining waste; biological samples, e.g., lysates prepared from crops or tissue samples; manufacturing samples, e.g., time course during preparation of pharmaceuticals; as well as libraries of compounds prepared for analysis; and the like. Samples of interest include compounds being assessed for potential therapeutic value, e.g., drug candidates from plant or fungal cells.

The term “samples” also includes the fluids described above to which additional components have been added, for example, components that affect the ionic strength, pH, or total protein concentration. In addition, the samples may be treated to achieve at least partial fractionation or concentration. Biological samples may be stored if care is taken to reduce degradation of the compound, e.g., under nitrogen, frozen, or a combination thereof The volume of sample used is sufficient to allow for measurable detection, usually from about 0.1 micron to 1 ml of a biological sample is sufficient.

Compounds and candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, naturally or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification to produce structural analogs.

### Output Variables

Output variables are quantifiable elements of cells or biological processes, particularly elements that can be accurately measured in a high throughput system. An output can be any cell component or biological product including, e.g., viability, respiration, metabolism, metabolite, cell surface determinant, receptor, micronuclei formation, protein or conformational or posttranslational modification thereof, lipid, carbohydrate, organic or inorganic molecule, mRNA, DNA, or a portion derived from such a cell component. While most outputs will provide a quantitative readout, in some instances a semi-quantitative or qualitative result will be obtained. Readouts may include a single determined value, or may include mean, median value or the variance. Characteristically a range of readout values will be obtained for each output. Variability is expected and a range of values for a set of test outputs can be established using standard statistical methods.

Various methods can be utilized for quantifying the presence of the selected markers. For measuring the amount of a molecule that is present, a convenient method is to label the molecule with a detectable moiety, which may be fluorescent, luminescent, radioactive, or enzymatically active. Fluorescent and luminescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g., by expressing them as green fluorescent protein chimeras inside cells.

Output variables may be measured by immunoassay techniques such as, immunohistochemistry, radioimmunoassay (RIA) or enzyme linked immunosorbance assay (ELISA) and related non-enzymatic techniques. These techniques utilize specific antibodies as reporter molecules that are particularly useful due to their high degree of specificity for attaching to a single molecular target. Cell-based ELISA or related non-enzymatic or fluorescence-based methods enable measurement of cell surface characteristics. Readouts from such assays may be the mean fluorescence associated with individual fluorescent antibody-detected cell surface molecules or cytokines, or the average fluorescence intensity, the median fluorescence-intensity, the variance in fluorescence intensity, or some relationship among these.

The results of screening assays may be compared to results obtained from reference compounds, concentration curves, controls, etc. The comparison of results is accomplished by the use of suitable deduction protocols, AI systems, statistical comparisons, etc. A database of reference output data can be compiled. These databases may include results from known agents or combinations of agents, as well as references from the analysis of cells treated under environmental conditions in which single or multiple environmental conditions or characteristics are removed or specifically altered. A data matrix may be generated, where each point of the data matrix corresponds to a read-out from a output variable, where data for each output may come from replicate determinations, e.g., multiple individual cells of the same type. The readout may be a mean, average, median or the variance or other statistically or mathematically derived value associated with the measurement. The output readout information may be further refined by direct comparison with the corresponding reference readout. The absolute values obtained for each output under identical conditions will display a variability that is inherent in live biological systems and also reflects individual cellular variability as well as the variability inherent between individuals.

Alternative in vivo uses for the system include implantation into a subject for experimental studies, to provide assistance for impaired functions, to augment natural functions, or to provide extra capabilities.

As mentioned previously, the present invention also relates to systems and methods of a microplasm functionalized surface patterning of a substrate. The present invention represents an improvement over existing plasma systems used to modify the surface of a substrate, as the present invention creates surface patterning without the use of a mask, stamp or a chemical treatment.

In some embodiments, the microplasm functionalized surface patterning of a substrate is used in conjunction with a cell printing system and method. When used in combination with a cell printing system, the microplasm systems and methods of the invention create patterned cells on various substrates without using a mask, a stamp or a chemical treatments.

In other embodiments, the microplasm functionalized surface patterning of a substrate is used in conjunction with biomolecule printing system and method. When used in combination with a cell printing system, the microplasm systems and methods of the invention create patterned biomolecules on various substrates without using a mask, a stamp or a chemical treatments.

In the microplasma functionalized surface patterning, an atmospheric pressure, low-temperature microplasma is generated with a dielectric barrier discharge (DBD) plasma system consisting of a micro-second pulsed power supply and electrode system. By using the microplasma surface patterning systems and methods of the present invention, when integrated with a cell and/or biomolecule bioprinting system, chemically and physically predesigned patterns of cells and/or biomolecules can printed onto a functionalized patterned substrate surface with precise spatial positioning.

### Precision Extrusion Deposition

A PED useful in the systems and methods of the present invention has been previously described (Wang, et al., 2004, Rapid Prototype Journal, 10:1, 42-49; see also U.S. application Ser. No. 11/842,796) that forces powder or pellets of material, such as polycaprolactone, through a heating element where it is melted and extruded out from a microscale nozzle with pressure generated by a rotating screw. The extruded materials are guided by nozzles and solidified as strands of small diameter. Mounted on a 3D positioning system, the extrusion head may deposit these strands at any width, of fill gap, apart from each other. Once a layer is complete, the extrusion head is moved upwards one increment, or layer height, and more strands are deposited at a variable angle to the previous layer. The control of fill gap allows fine control of porosity. The control of fill gap and layer height allows fine control of pore size. The process can use polycaprolactone as well as other polymers for which the phase transition can be controlled through the extruding deposition.

### Microplasma System

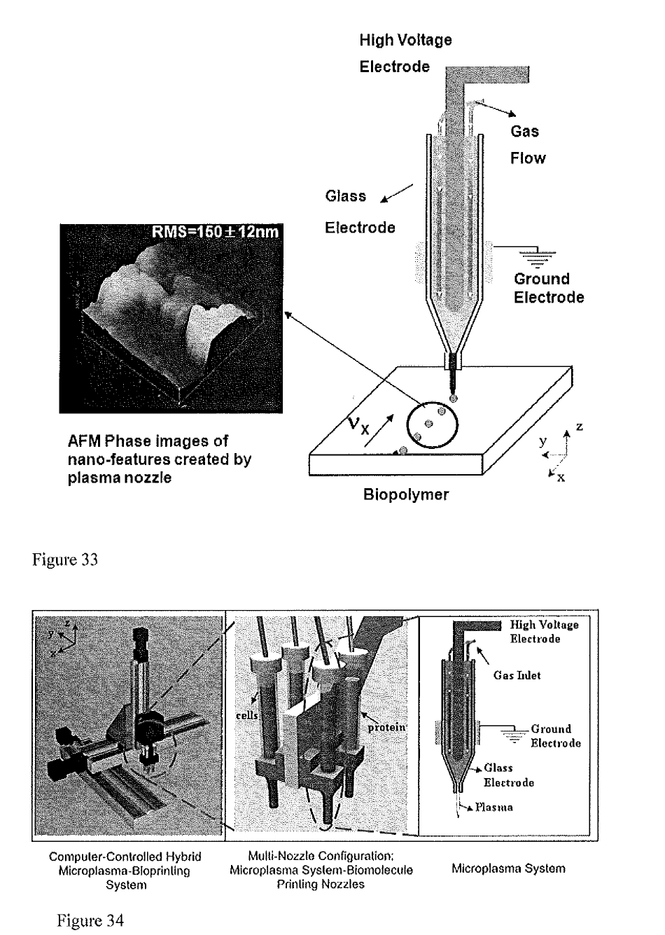

In one embodiment, the apparatus comprises a microplasma nozzle that is fixed adjacent to a substrate material that is affixed to a platform moveable by a motion control system to position and move the platform in the X, Y and Z directions in relation to the fixed microplasma nozzle to create the desired functionalized pattern on the surface of the substrate material (See, for example, FIG. 33). In another example embodiment, the apparatus comprises a moveable microplasm nozzle affixed to motion control system to position and move the microplasma nozzle in the X, Y and Z directions in relation to a substrate material to create the desired functionalized pattern on the surface of the substrate material (See, for example, FIG. 34).

In additional embodiments, the microplasma nozzle is integrated into a multi-nozzle bioprinting system. A suitable multi-nozzle bioprinting system comprises multiple nozzles of different types and sizes, including at least one microplasma nozzle for creating functionalized patterning on the surface of a substrate, thus enabling the deposition of a substrate material having different microplasma functionalized surfaces, to, for example, create three-dimensional tissue scaffolds.

In a preferred embodiment of the multi-nozzle bioprinting system, at least four types of nozzles are used in the system, with at least one nozzle being a microplasma nozzle for creating functionlized patterns on the surface of the substrate. Example nozzle types include, but are not limited to, microplasma nozzle, solenoid-actuated nozzles, piezoelectric glass capillary nozzles, pneumatic syringe nozzles, and spray nozzles, with size ranges varying from about 30 pm to about 500 pm. The multiple nozzle capability allows for the serial or concurrent deposition of cells, growth factors, and scaffold materials having desired functionalized patterns, thus enabling the construction of heterogeneous scaffolds with bioactive compounds, or establishing functional gradient scaffolds with different mechanical/structural properties in different scaffold regions. An example of a multi-nozzle bioprinting systems and its methods of use adaptable for use with the microplasma nozzle of the invention is described in U.S. application Ser. No. 10/540,968, incorporated by reference herein in its entirety.

Alternative nozzles or other devices can also be used to provide various substrate coatings, washings or functionalities. For example, biochemical surface treatment can be performed via a nozzle or other device, for example, by washing, spraying, etc., simultaneously with the deposition of scaffolding materials through another nozzle. A coating material can also be sprayed on the device simultaneously with the deposition of the scaffolding material through another nozzle, or a coating material can be sprayed onto a single layer or layers of the device. An additional nozzle or other device can also be used to add a support material or temporary scaffolding that can later be removed from the finished part, for example, a reversible gel, simultaneously with the deposition of the scaffolding material through another nozzle.

A nozzle affixed to the device can also be used to deliver energy to modulate the scaffold solidification, for example, to transmit UV or laser energy through an optical fiber simultaneously with the deposition of the scaffolding material through another nozzle. An additional nozzle can also be used to deposit, extrude or pattern electrically conductive materials within the scaffold simultaneously with the deposition of the scaffolding material through another nozzle to generate wired, circuited, or biochip embedded scaffolds. An additional nozzle can also be used to transmit/deposit fluid simultaneously with the deposition of the scaffolding material through another nozzle. The fluid can be applied to the part for various purposes such as cooling, sterilization, cross-linking, solidification, etc.

In-situ sterilization can be incorporated into the method of the present invention as well and can be done in several ways. In one embodiment, a solution with antibiotics such as penicillin is added through the multi-nozzle deposition system while making the device or afterwards. In another embodiment, a sterilizing solution (non-antibiotic) is added to one of the nozzles for deposition or post-sterilization. An alternative device to a nozzle, as part of the multi-nozzle deposition system, can also be used such as device emitting ultraviolet radiation, heat, or gamma irradiation.

The method and system of the present invention may further comprise imaging capabilities such as an ultrasonic transducer that can be used for imaging the device while it is being built. Alternatively, an optical imaging apparatus, such as a microscope, can be used to provide visual information, or provide data for feedback in a closed-loop control system. An optical imaging apparatus can also be used to monitor fluorescence and reporter gene activities which can be used for cell counting, calculating the presence of proteins, DNA expression, metabolic activity, cell migration, etc. Atomic force microscopy and scanning tunneling microscopy, can also provide information about the device at nanoscale resolution.

Sensing devices can also be incorporated into the methods and system to provide relevant data such as temperature, or to monitor chemical reactions, chemicals released during production, and/or mechanical forces such as shear during production. Such sensing devices can be used to create a feedback control mechanism to regulate the process parameters in an automated fashion.

Mechanical agitation or stimulation devices such as ultrasonic, subsonic, and/or sonic transducers can also be incorporated into the methods and system to stimulate the device mechanically during construction. The stimulations will help to improve the device structural properties, for example, homogeneity of the cell and scaffolding material distribution.

### Scaffold Materials

Non-limiting examples of material useful as scaffold materials include, but are not limited to, poly-capralactone, poly-lactic acid, poly(lactic-co-glycolic acid) (PLGA), tricalcium phosphate, hydroxyapatite, polyglycolic acid, polyhydroxybutyrate, and polypropylene fumarate, poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), nylons, polyamides, polyanhydrides, poly(ethylene-co-vinyl alcohol), poly(vinyl acetate), poly(ethylene oxide) (PEO) and polyorthoesters, alone or in combination with other materials. The scaffold materials are preferably biocompatible. The scaffold material can have a wide range of biodegradability, depending on the desired properties and purpose of the scaffold.

The scaffold material can also be combined with various additives to better suit the type of cell or tissue that is being used. By way of one non-limiting example, hydroxyapatite could be used when working with osteoblasts to create bone implant scaffolds. The scaffold could also be coated with biomolecules, such as proteins, growth factors, and/or receptors, that provide useful cues, such as facilitating cellular adhesion or migration onto the scaffold surface.

In some embodiments, the scaffold material can optionally include a hydrogel, such as, by way of non-limiting examples, alginate, collagen, chitosan, fibrin, hyaluronic acid, agar, polyethylene glycol and its copolymers, and acrylamide-based and acrylic acid-based polymers.

In various embodiments, the systems and methods of the invention can be used to improve a scaffold's properties to better imitate the structure and function of ECM, to coat the scaffold with biomolecules to supply sufficient biological cues, and to introduce additional chemical-groups and/or physical features to the scaffold to provide particular chemical and/or physical cues.

In various embodiments, the systems and methods of the invention can be used to modify surface roughness. As disclosed herein, plasma modification was demonstrated to increase surface roughness dramatically, for example to the roughness value of 150+12 nm, a value that is almost 4 times higher than the unmodified surface roughness value.

In various embodiments, the systems and methods of the invention can be used to modify the physiochemical surface properties of a scaffold to affect the cell function, such as, cell attachment, cell proliferation and cell differentiation.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

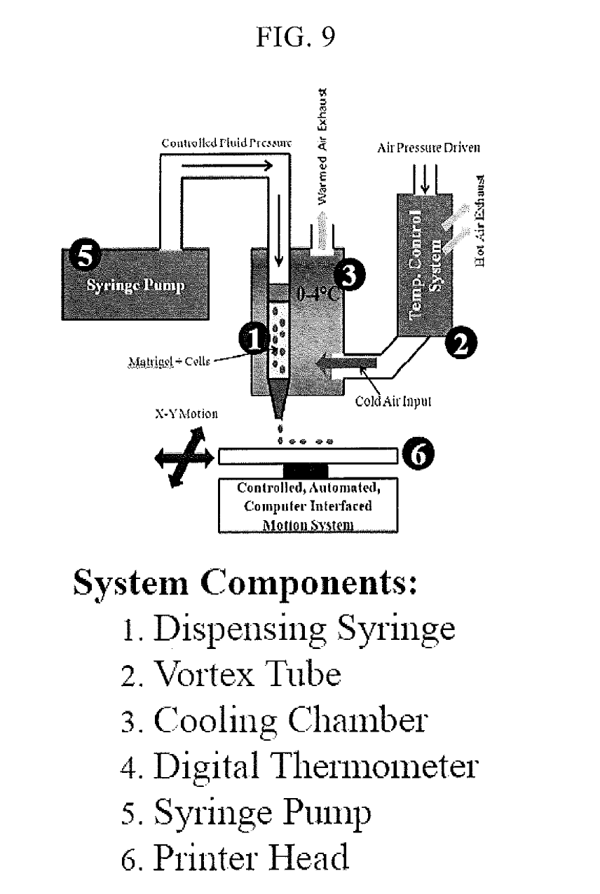

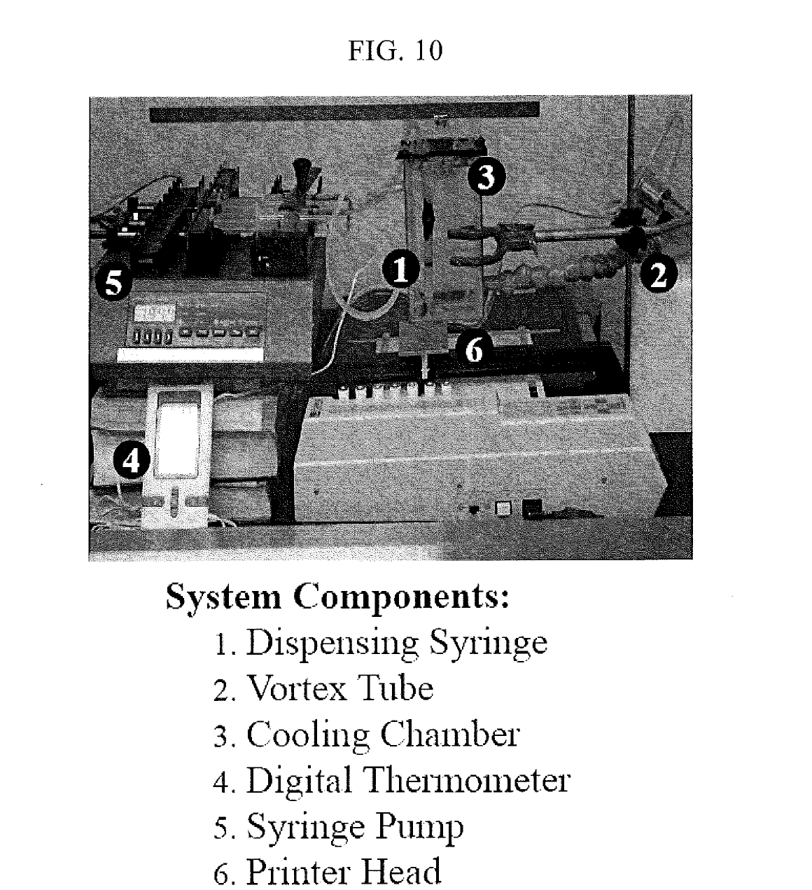

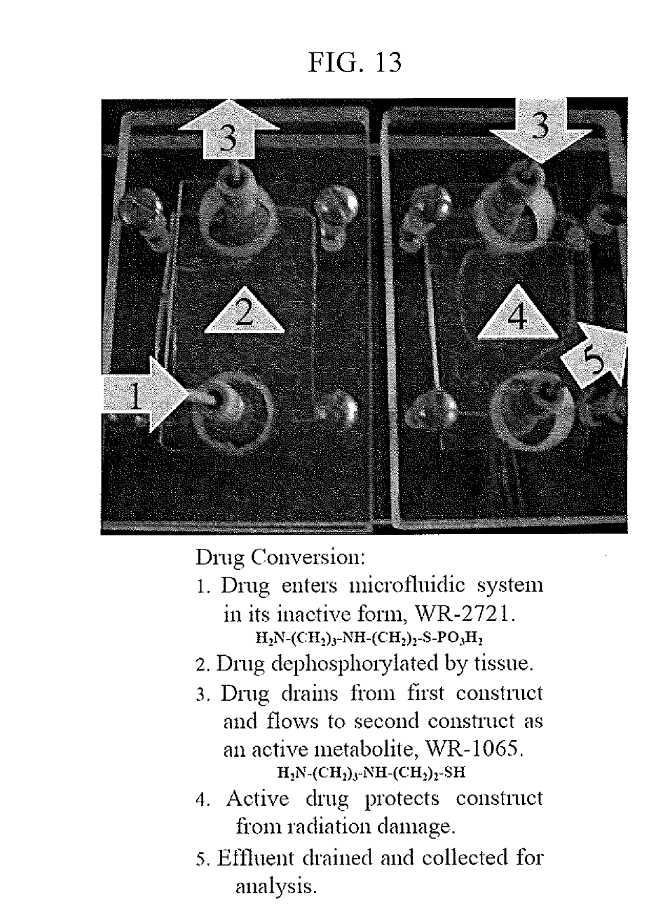

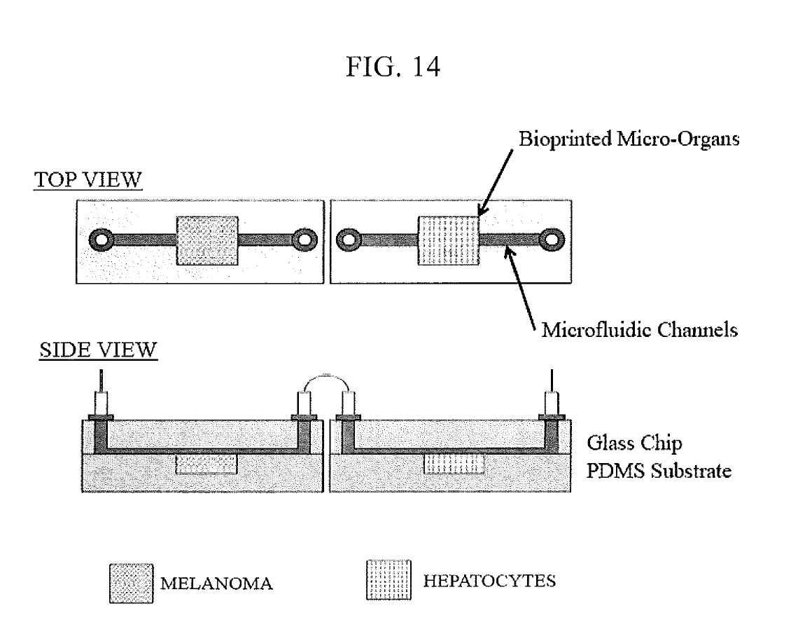

Human hepatocytes (HepG2) and human melanoma cells (M10) were cultured in α-Minimum Essential Medium (Gibco) at 37° C. in 5% CO₂. Cells were mixed with BME (i.e., Basement Membrane Matrix Phenol Red-free (BD Biosciences Matrigel)) chilled to 4° C. and printed onto a glass microfluidic chip having a PDMS substrate (see FIGS. 13 and 14) using a temperature-controlled BME printing apparatus 0-5° C. (see, for example, FIGS. 9 and 10).

After 24 hours, cells were treated with an inactive form of the anti-radiation drug amifostine (see Grochova, 2007, J Appl Biomed 5:17) by perfusion of a 1 mM solution of the inactive form of amifostine in media for 4 hours. The inactive form of amifostine (i.e., WR-2721; H₂N-(CH₂)₃-NH—(CH₂)₂-S—PO₃H₂) is converted to the active form (i.e., WR-1065; H₂N—(CH₂)₃—NH—(CH₂)₂—SH) when it is dephosphorylated by cells. After the 4 hour treatment with amifostine, the cells were exposed to 2 Grays of γ-radiation from a cesium source. Immediately after exposure to radiation, cells were again treated by perfusion with a 1 mM solution of the inactive form of amifostine in media for 90 minutes. Then, the cells were incubated and allowed to divide for 60 hours and examined for evidence of radiation damage.

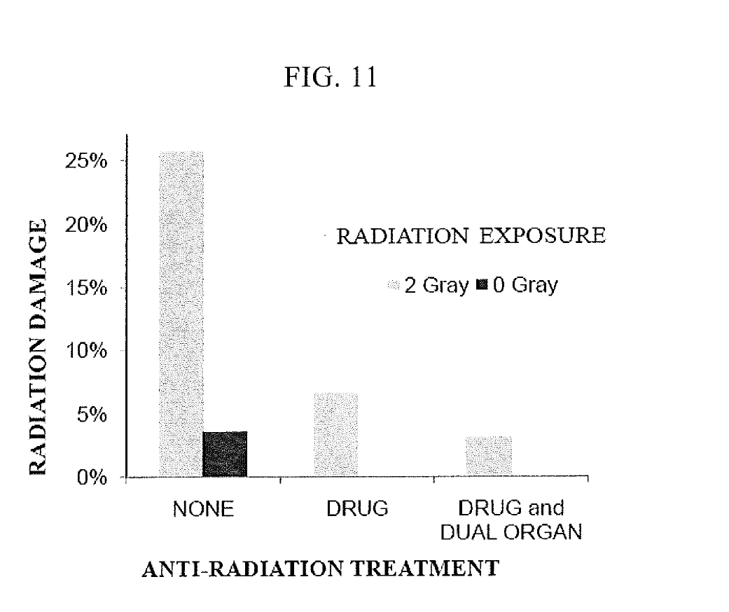

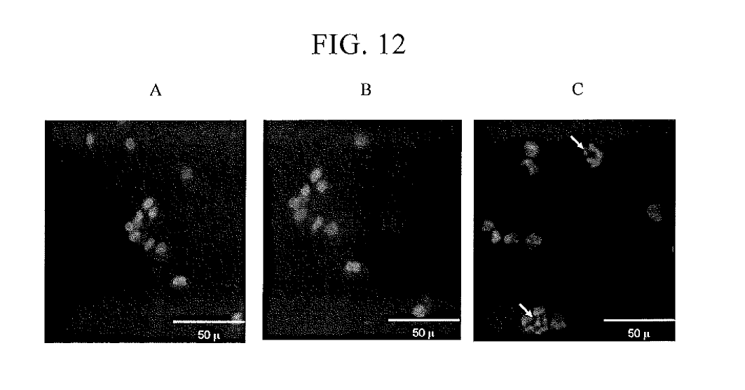

Binucleated cells were examined for the presence of micronuclei as evidence of radiation damage. Of the radiation-treated cells not treated with amifostine, 26% were observed to have micronuclei (see FIG. 11). Of the radiation-treated cells treated with amifostine in a tissue culture plate, about 4% were found to have micronuclei. Of the radiation-treated cells treated with amifostine in the microfluidic chip, only about 3% were found to have micronuclei, which was comparable to the control cells not treated with radiation or amifostine.

### Example 2

### Plasma Treatment of Polycaprolactone Scaffolds

This example describes a solid free-form fabrication (SFF) technology based Precision Extrusion Deposition (PED) process for manufacturing manufacture three-dimensional (3D) polycaprolactone scaffolds and their surface treatment with plasma source for enhanced osteoblast cell adhesion and proliferation. The PED process allows the manufacture of tissue engineering scaffolds based on designed geometry with complete interconnectivity, controllable porosity. The as-fabricated polycaprolactone scaffolds have a 0/90° strut configuration of 300 μm pore size and 250 μm strut width. In order to improve cellular activity on 3D polycaprolactone scaffolds, they were surface treated with oxygen-based plasma source. The surface hydrophilicity and total surface energy of polycaprolactone was increased with plasma treatment. Comparison was made between different plasma treatment times, including 30 seconds, 1, 2, 3, 5 and 7 minutes to identify the plasma treatment duration suggesting higher cellular adhesion and proliferation. The maximum value of total surface energy and its components (polar and dispersive) was observed in 3-min treated polycaprolactone scaffolds. In addition, the positive effect of plasma treatment was observed in strength of cell adhesion which was increased 55% on 3-min plasma treated scaffolds compared to untreated and other plasma treatment duriations. The cell culture study over 7 days period also showed that the cell number on 3-min treated scaffolds is threefold of the number of cells on untreated scaffolds.

The Materials and Methods used in Example 2 are now described.

### Scaffold Manufacturing

Polycaprolactone was used as a scaffold material (Sigma-Aldrich Chemical Co). The mechanical properties of polycaprolactone (Mn=42,000) used in Example 2 had a tensile modulus of 400 Mpa with elongation at yield of 7.0 percent.

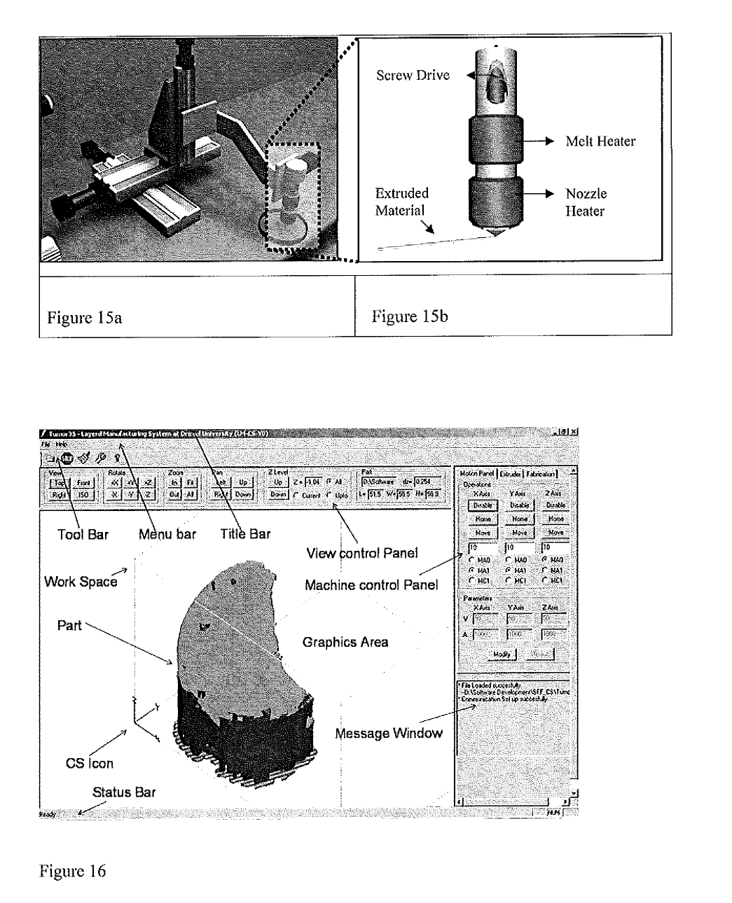

The scaffolds used in Example 2 were manufactured by a Precision Extrusion Deposition (PED) System. PED is based on Solid Free Form technique that can build physical model by extruding the material layer-by layer according to the designed geometry. The PED consists of hardware and software systems. The hardware system is called Material Deposition System where XYZ position system, material extrusion system, and a temperature control system located. The schematic of hardware system components are given in FIG. 15.

Prior to scaffold fabrication, the CAD based design geometry of the scaffold needs to be introduced to the system. It was conducted by the software system called Motion Control System (MCS), consisting of data processing software and system control software. In data processing software, the geometry was converted into STL format and then sliced while each slice pattern stored in the pattern library for toolpath generation. In system control software, the material deposition parameters were controlled according to the generated toolpath. The interface of the system control software is depicted in FIG. 16.

After the geometry was introduced to the system and process parameters were defined, the material extrusion system first melted the pellet of polycaprolactone to form a biopolymer and extruded it through a nozzle. The pellet form material was fused by two heating bands called melt heater and nozzle heater bands (FIG. 15b). The temperature of these two bands was adjusted via a temperature control system. The melt heater was kept at 110° C. while the nozzle heater was adjusted to 90° C. for the particular material used in this study. The melted material was delivered to the tip of the nozzle through pressure created by a turning precision screw (FIG. 15b). The velocity and the position of the nozzle can be defined through system control software (FIG. 16).

### Plasma Treatment of Scaffolds

The scaffolds were treated with a plasma reactor (PDC 32G, Harrick Scientific Inc., New York) for various time intervals. The system included a radiofrequency generator capable of 0-18 W at frequency range of 8-12 MHz, a vacuum pump, a helical internal electrode around the reactor, and instrumentation for pressures. The scaffolds were placed inside the chamber and exposed to the oxygen-based plasma for 30 seconds, 1, 2, 3, 5 and 7 minutes. The flow rate of oxygen was 1 standard liter/min. Following the plasma treatment, the samples were moved to a laminar hood for further surface and cell-scaffold interaction characterizations.

### Surface Characterization

The surface hydrophilicity and solid surface energy was assessed. The contact angle measurement was conducted by evaluating the effect of oxygen-based plasma treatment on polycaprolactone in terms of degree of hydrophilicity and solid surface energy. The contact angle (θ) of probe liquids were measured on plasma treated and untreated samples after dropping of probe liquid (2 μL) onto the surface. The measurements were taken at least four times to obtain a grand average.

The measured contact angles (θ) were used to calculate the solid surface energy (σₛ) of the plasma treated and untreated polycaprolactone surfaces based on Young's Equation (Volpe et al., 2002, Colloids and Surfaces a-Physicochemical and Engineering Aspects 206: 47-67).

σₛ=γₛₗ+σₗ·cos θ

In above equation (1), the surface energy of probe liquids (σₗ) can be obtained from the literature for different probe liquids (Zenkiewicz, 2005, International Journal of Adhesion and Adhesives 25:61-66), and the solid/liquid interfacial energy (γₛ₁) for polymers can be calculated based on Owens-Wendt method (Wu, 1971, Journal of Polymer Science Part C 34). In the Owens-Wendt's method, the surface tension of liquid and solid phase are the sum of theirs dispersion component (σ^{D}) and the polar component (σ^{P})

$$\begin{matrix}{σ}_{l} = {σ}_{l}^{P} + {σ}_{l}^{D} {σ}_{s} = {σ}_{s}^{P} + {σ}_{s}^{D} & ( 2 ) \\ {γ}_{sl} = {σ}_{s} + {σ}_{l} - ( \frac{4 {σ}_{l}^{D} {σ}_{s}^{D}}{{σ}_{l}^{D} + {σ}_{s}^{D}} + \frac{4 {σ}_{l}^{P} {σ}_{s}^{P}}{{σ}_{l}^{P} + {σ}_{s}^{P}} ) & ( 3 )\end{matrix}$$

where superscripts D and P represent the dispersive and polar components, subscripts s and 1 denote solid and liquid phases, respectively. By substituting interfacial tension (γₛₗ) into Young's Equation (Eq.1) it is possible to calculate the polar and disperse fractions of the surface energy with the aid of a single linear regression from the contact angle data of various liquids.

Three different probe liquids, diiodomethane (Fisher, PA), glycerol (Fisher, PA), and ultra pure water (Agilent, Germany) were used. Surface energy components of these probe liquids (σₗ^{P}, σₗ^{D}) are given in Table 1.

TABLE 1
Liquid surface tension (mN/m) and its dispersive and polar fractions
for probe liquids

| | | | Dispersive |
|---|---|---|---|
| | Surface | Polar Component | Component |
| | Tension | of Liquid Surface | of Liquid Surface |
| Probe Liquids | of Liquid (σₗ) | Tension (σₗ^{P}) | Tension (σₗ^{D}) |
| Ultrapure Water | 72.88 | 50.4 | 21.6 |
| Glycerol | 63.30 | 43.1 | 20.2 |
| Diiodomethane | 50.00 | 2.3 | 47.7 |

### Cell-Scaffold Interaction

The effect of plasma on cell-scaffold interaction was investigated through culturing 7F2 mouse osteoblast cells (CRL-12557, American Type. Culture Collection, Rockville, Md.) on plasma treated and untreated samples for 7 days. The cells with a passage number 23-30 were used in current study. The cells were cultured in alpha-minimum essential medium (α-MEM) (Gibco, N.Y.) containing 10% fetal bovine serum (Hyclone, Utah), 2 mM L-glutamine and 1 mM sodium pyruvate without ribonucleosides and deoxyribonucleosides in an incubator at 37° C. and 5% CO₂. Upon confluency, 7F2 cells were trypsinized and resuspended in medium with different concentrations for further cell/scaffold interaction studies including assessment of cell adhesion and cell proliferation.

### Measurement of Cell Adhesion

A shear flow assay was used for quantifying the strength of 7F2 cell adhesion on untreated and plasma treated polycaprolactone surfaces with controlled detachment force. The main assumption in this assay was that the shear stress at the wall (τ_{w}=6 μQ/wh²) was equal to detachment shear stress at the surface of the cells. Before starting to measure cell adhesion strength, 1 mL cell suspension with a concentration of 2.0×10⁴ cells/mL was seeded onto the polycaprolactone samples and permitted to settle and attach for two hours in the incubator. After the incubation, the sample was placed inside the flow chamber (25×75×1 mm) (w×L×h) and the medium was initiated between the plates with 100-200 ml/min for 1 min to obtain the effective shear stress (τ₅₀) at which 50% of the seeded cells were removed compared to the control sample. After determining the effective shear stress, it was applied to all polycaprolactone samples plasma treated for 30 seconds, 1, 2, 3, 5 and 7 minutes to identify the plasma treatment duration creating surface that enhances the cell attachment superior than other treatment durations. Following the flow, the attached cells remained on the surface of interest were counted by alamarBlue™ assay (Biosource International, USA).

### Measurement of Cell Proliferation

The cell proliferation on untreated and oxygen-plasma treated 3D polycaprolactone scaffolds was quantified with alamarBlue™ (aB) assay (Biosource International, USA). It is non-toxic indicator of oxidation-reduction mechanism associated with the a shift in color of the culture medium from blue to fluorescent pink (Sherry et al., 1998, In Vitro Cellular & Developmental Biology—Animal 34:1543). The produced color product can be read by microplate fluorometer and the amount of fluorescence is directly proportional to the number of living cells. To conduct the cell proliferation study, 1 mL cell suspension with a concentration of 2.0×10⁴ cells/mL was seeded on untreated and plasma treated polycaprolactone samples. Then samples were incubated at 37° C. and 5% CO2 condition until the characterization day. At the day of characterization, 10% (v/v) of aB assay solution was added to each well and allowed to incubate for 4 hours at 37° C. and 5% CO₂ condition. After incubation, 800 μL solution was taken out of each well to measure the fluorescence intensity by microplate fluorometer (Genius, TECAN, USA) at 535 nm excitation and 560 nm emission wavelengths.

The Results of Example 2 are now described.

### Fabrication of 3D Polycaprolactone Scaffolds

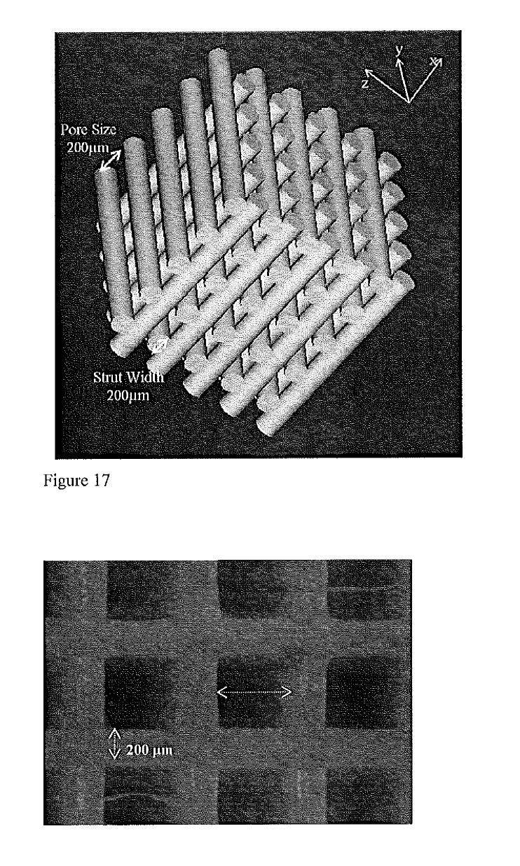

Prior to fabricating the scaffolds used in this Example, the PED machine was tested to identify the resolution of the PED system by measuring the difference between the designed and manufactured one. For that purpose, a scaffold designed with CAD system. The specification of the designed scaffold is given in FIG. 17.

Based on the designed model, the toolpath was defined to the PED System and 10 scaffolds were manufactured. Then for each scaffold, five measurements were taken from five different parts of the scaffold and grand average were taken for strut width and pore size values. The measurement data is given in Table 2.

TABLE 2
The strut width and pore size measurements of ten scaffolds
manufactured by PED.

| Scaffold | Strut Width (μm) | Pore size (μm) |
|---|---|---|
| Scaffold 1 | 216 ± 8 | 199 ± 7 |
| Scaffold 2 | 217 ± 4 | 195 ± 5 |
| Scaffold 3 | 211 ± 6 | 187 ± 7 |
| Scaffold 4 | 228 ± 5 | 173 ± 3 |
| Scaffold 5 | 213 ± 9 | 212 ± 6 |
| Scaffold 6 | 207 ± 5 | 218 ± 6 |
| Scaffold 7 | 223 ± 10 | 214 ± 9 |
| Scaffold 8 | 222 ± 6 | 184 ± 11 |
| Scaffold 9 | 239 ± 3 | 158 ± 6 |
| Scaffold 10 | 220 ± 7 | 198 ± 11 |

The ± represents the standard deviation of five different measurements from scaffolds.

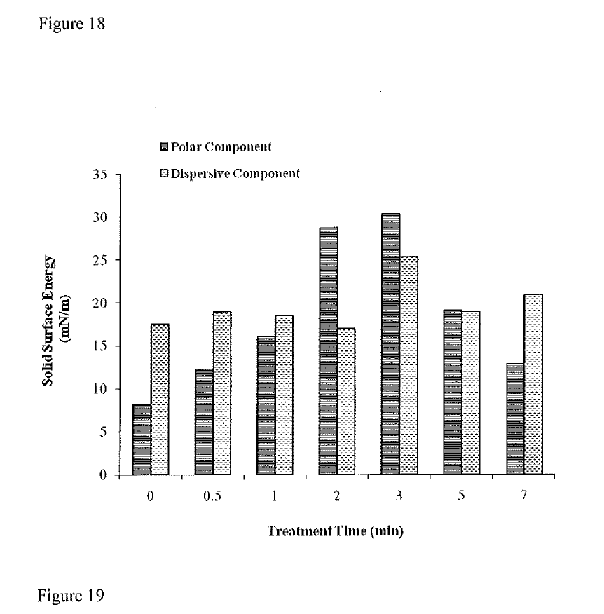

Based on the measurements of data given in Table 2, the average strut width for 10 different scaffolds is 219.66 μm with a standard deviation of 9.14 μm. This corresponds to accuracy of 90% based on designed 200 μm strut width. For the pore size, the average value is 194.01 μm with a standard deviation of 5.42 μm based on measurements taken from 10 scaffolds. For the 200 μm designed pore size the average value corresponds to 97% accuracy which is highly precise compared to the other solid free-form scaffold manufacturing techniques. Following by the resolution analysis of the system, the scaffolds used in current study were designed and manufactured with a 0/90° strut configuration of 300 μm pore size and 250 μm strut width. The top view of the scaffold use din this study is given in FIG. 18.

### Effect of Plasma Treatment on Surface Hydrophilicity and Energy

The surface wettability (surface hydrophilicity) was quantified by conducting contact angle measurements with polar and non-polar probe liquids. Independent from plasma source and substrate type, generally the oxygen containing plasma enhances the surface hydrophilicity. Nevertheless, depending on each system configuration, the exposure time to plasma may affect the degree of surface hydrophilicity. In the present Example, the contact angle on a polycaprolactone surface exposed to plasma was measured after various time durations to identify the efficient plasma treatment duration for polycaprolactone samples for hydrophilicity under oxygen-based low pressure plasma. The contact angle measurements of probe liquids on polycaprolactone samples exposed to various plasma treatment durations are given in Table 3.

TABLE 3
The static contact angle data of probe liquids on polycaprolactone
surface exposed to oxygen plasma for 0, 0.5, 1, 2, 3, 5 and 7 minutes.

| Treatment | Static Contact Angle(°) | | |
|---|---|---|---|
| Duration | Ultra Pure Water | Glycerol | Diiodomethane |
| 0 min | 59 ± 6 | 63 ± 3 | 30 ± 7 |
| 0.5 min | 35 ± 3 | 50 ± 3 | 27 ± 2 |
| 1 min | 23 ± 2 | 36 ± 4 | 27 ± 4 |
| 2 min | 21 ± 3 | 39 ± 3 | 16 ± 4 |
| 3 min | 18 ± 1 | 30 ± 2 | 10 ± 1 |
| 5 min | 23 ± 1 | 42 ± 3 | 16 ± 2 |
| 7 min | 22 ± 2 | 33 ± 3 | 11 ± 2 |

The ± represent standard deviation with n = 4 for each plasma treatment time and for each probe liquids.

In Table 3, contact angles are given for the three probe liquids ultrapure water, diiodomethane, and glycerol. It is apparent from the table that the contact angles decrease significantly even after 30 seconds plasma exposure. The decline observed constantly for 1, 2 and 3 minute plasma treated samples. The minimum contact angle value was observed for 3-min plasma treated samples. The contact angle value from untreated (0 min) to 3-min plasma treated samples decrease 69% for ultrapure water, 52% for glycerol and 67% for diiodomethane. However, after 3-min plasma treatment, the contact angle starts to increase with the plasma treatment time. The samples treated with 5 min and 7 minutes plasma showed increment in contact angle with 28%, 40% and 60% for ultrapure water, glycerol and diiodomethane, respectively.

The total surface energy (σₛ) of polycaprolactone and its polar (σₛ^{P}) and dispersive (σₛ^{D}) components before and after oxygen plasma treatment, were calculated by using Owens-Wendt's method. In FIG. 19, the changes in polar, dispersive components of solid surface energy and total surface energy surface with plasma treatment time are given. On 3-min plasma treated samples both polar and dispersive components showed the highest value when compared to other treatment time durations. Polar component of the surface energy followed the same trend with contact angle measurement data (FIG. 19). It was increased with the plasma treatment time until 5-min treatment and then decreased dramatically. On the other hand, the dispersive component of surface energy did not show significant changes with the plasma treatment. One can say that polar component was affected by the plasma when compared with the dispersive component.

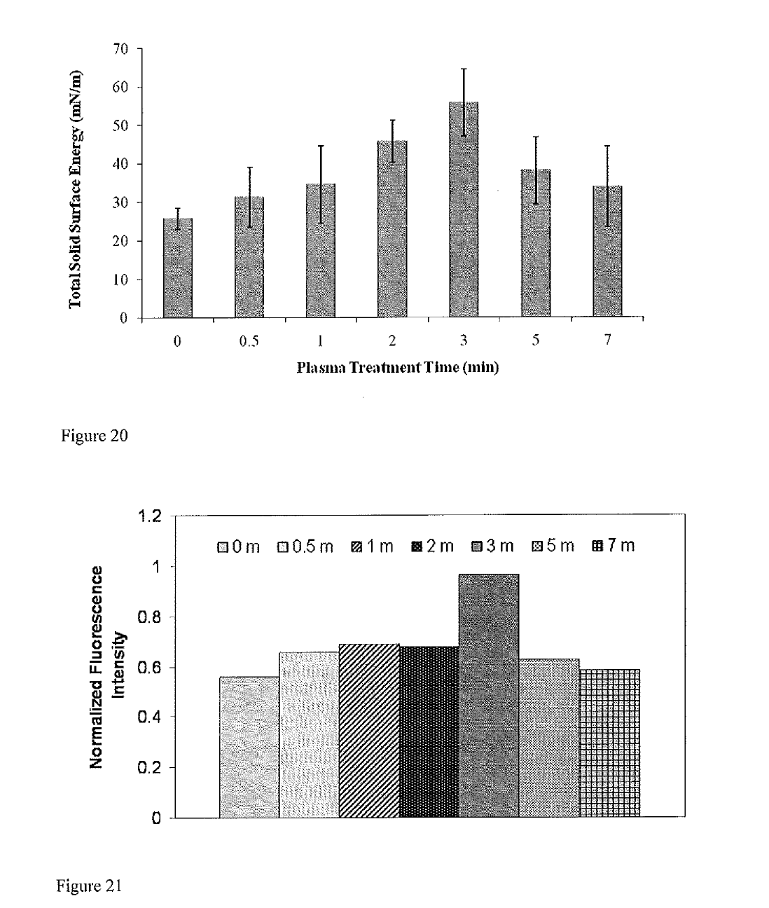

The total surface energy change of polycaprolactone with plasma treatment was given in FIG. 20. From the Owens-Wendt's method we knew that the total surface energy is sum of polar and dispersive component. The trend in FIG. 20 shows that the total surface energy increased with plasma treatment time until 5-min plasma treatment and decreased as observed in polar component. FIGS. 19 and 20 indicate that polar component in total surface energy is dominant factor compared to dispersive for polycaprolactone samples exposed to oxygen-based plasma treatment.

The increment in total surface energy until certain treatment time duration can be explained through the etching effect of plasma. Once the oxygen-based plasma contact with the polymer, first it is breaking the C—C and C—H bonds at the backbone and introduces oxygen containing polar functional groups being responsible in surface polarity. With the increment in treatment duration the ions, electron, excited species start to etch the surface and break the oxygen functional groups and eventually introducing apolar C—C and C—H groups to the surface. This increase the apolarity causes increase in contact angle on the surface which leads to decline in polar component of the surface energy.

### Effect of Plasma Treatment on Cell Attachment and Proliferation

The strength of cell adhesion on plasma treated surface for 0, 0.5, 1, 2, 3, 5 and 7 minutes was measured by shear flow assay. In this assay, for polycaprolactone samples the effective shear stress (τ₅₀) was calculated as 27 dynes/cm². The flow was initiated corresponding to effective shear stress and cell number on surface of interest was calculated by alamarBlue™ 535 nm excitation and 560 nm emission wavelengths. FIG. 21 demonstrates the normalized fluorescence intensity of attached cells on untreated (0 min) as well as 0.5,1,2,3,5, and 7 min plasma treated polycaprolactone sample. Normalized fluorescence intensity was determined through dividing the cell number on surface of interest exposed to effective shear stress by cell number on surface of interest without exposed any flow.

It is clear form FIG. 21 that the 3-min oxygen plasma treated had the greatest retention of cells at the same detachment force compared to 0, 0.5, 1, 2, 5, 7 minutes plasma treated samples. The cell number on 3-min treated scaffolds exposed to shear stress are almost equal to the cell number onto the control (without flow) samples. These results indicate cell adhesion influenced strongly by the hydrophilicity of the surface. Based on the surface hydrophilicity and surface energy data, we may conclude that increase hydrophilicity and surface energy result in increment in cell adhesion strength.

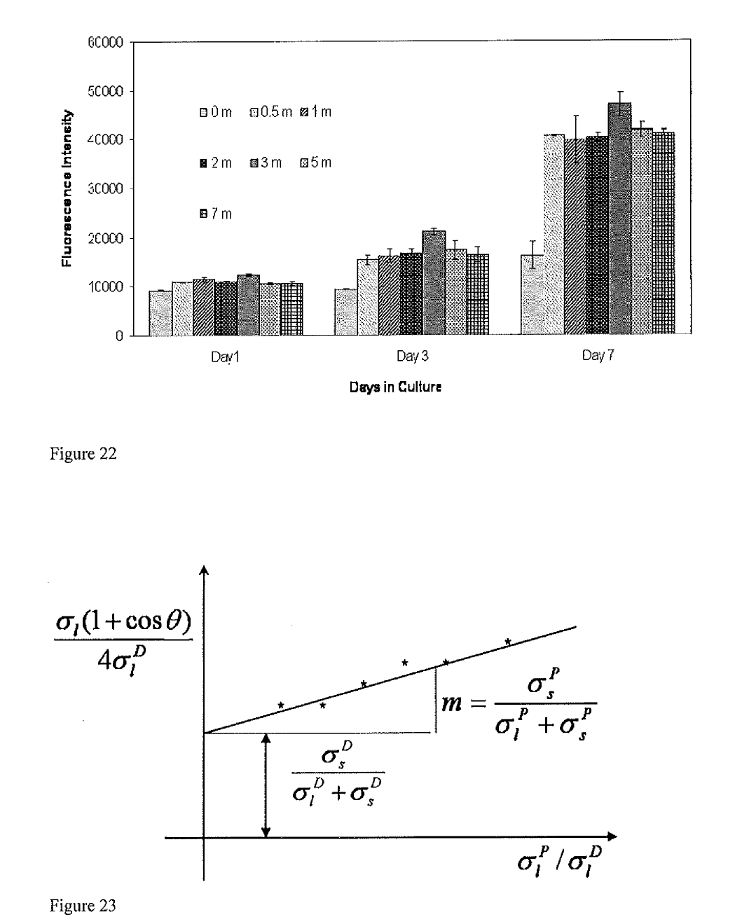

The cell proliferation on untreated and 3-min oxygen-plasma treated 3D polycaprolactone scaffolds were measured by alamarBlueTM assay at day 0, 3, and 7. FIG. 22 shows the cell proliferation on untreated and 30 second, 1, 2, 3, 5, 7 minutes treated polycaprolactone scaffolds. As seen in FIG. 22, there was an upregulation in fluorescence intensity for all sample groups except untreated polycaprolactone. Among them from day 1 to day 7, the cells on 3-min plasma treated scaffolds showed the highest fluorescence intensity. This result can be explained with the positive effect of cell attachment on cell proliferation. From cell attachment assay result (FIG. 21) we observed that cell attachment rate was almost 100% on 3-min plasma treated samples. When compared to the untreated polycaprolactone samples, cells on 3-min plasma treated scaffolds showed almost 50% higher cell adhesion strength under the same shear stress. While there was higher number of cells attached to 3-min treated scaffold, their proliferation rate on this particular scaffold was expected to be higher compared to the other samples.

### Example 3

### Cell-Scaffold Interaction

The combinatorial effect of protein coating and plasma modification on the quality of osteoblast-scaffold interaction was investigated. The three-dimensional polycaprolactone scaffolds manufactured by Precision Extrusion Deposition (PED) system were used. The structural, physical, chemical and biological cues were introduced to the surface through providing 3D structure, coating with adhesive protein fibronectin, modifying the surface with oxygen-based plasma. The changes in surface properties of polycaprolactone after being modified were examined by contact angle measurement, surface energy calculation, surface chemistry analysis (XPS), and the surface topography measurements (AFM). The effect of modification techniques on osteoblast short-term and long-term functions were examined by cell adhesion, proliferation assays and differentiation markers, namely alkaline phosphatase activity (ALP) and osteocalcin secretion. The results disclosed herein suggest that the presence of structural and chemical cues introduced by the PED fabrication, plasma modification and protein coating can greatly improve the cellular behavior of osteoblast in bone tissue formation.

The Materials and Methods used in Example 3 are now described.

### Three Dimensional Polycaprolactone Scaffold Fabrication

The complex structure of ECM can be imitated by manufacturing 3D tissue engineering scaffolds. Biofabrication techniques for implementing solid freeform fabrications (SFF) are capable of manufacturing 3D scaffolds in a layer-by-layer fashion from a three-dimensional computer design of the scaffolds. For example, Fused Deposition Modeling (FDM) (Hutmacher et al., 2001, Journal of Biomedical Materials Research 55:203-216; Zein et al., 2002, Biomaterials 23:1169-1178) and Precision Extrusion Deposition system (PED) (Shor, et al., 2009 Biomaterials 1: (1) 10; Wang, et al., 2004, Rapid Prototyping Journal, 10:42-49) are two commonly used additive manufacturing techniques that can fabricate 3D scaffolds with precise control over the macroscopic geometry, internal architecture, and interconnectivity. Even though both systems are able to manufacture complicated 3D structures based on pre-designed geometry, PED process has advantages in without using filament preparation. This unique feature provides benefit regarding the material choice, structural design and repeatability over the other tissue engineering scaffold manufacturing techniques (Shor et al., 2007, Biomaterials 28(35), 5291-5297).

In a PED process, biopolymers are extruded through a mini-turning screw out of the deposition nozzle. The biopolymers used in this system are typically thermoplastics with high tensile strength, making the scaffolds suitable candidates for hard tissue application. The PED system consists of software and hardware components. The software component contains two sub-systems, namely toolpath generation and motion controller software. In toolpath generation part, the solid scaffold model is transformed into 2D toolpath through converting the format into stereolithography (STL) format, and then slicing. The pore size, extruded strut diameter, and the layer height are key parameters in the scaffold configuration and these parameters can be defined into the toolpath generation program. The second sub-system is the motion controller software. The motion controller software is used to control the motion of the extrusion nozzle for material deposition. The parameters can be defined in motion controller software are, the speeds of X, Y and Z direction motion arms, and the starting position of the extrusion nozzle.

The PED hardware component contains XYZ positioning system, material extrusion system, and temperature control system. Once the process toolpath is introduced to the system, the XYZ positing system will set the extrusion nozzle at the starting point while the material extrusion system melts the pellet for fused extruding deposition. The melting process and temperatures are controlled by temperature control system which has controller and two thermo couples, namely the melt heater and nozzle heater. In current study poly-ε-caprolactone (Sigma Aldrich, Mo., USA) was used as a biopolymer for PED process. Polycaprolactone has a molecular with a molecular weight of 42,500 (Mn) with a melt index of 1.9 g/10 min (ASTM D-1238-73). Polycaprolactone was extruded in in 6×6×2 mm³ square geometry with 0/90° strut configuration had 300 μm pore size and 250 μm strut diameter. The PED melt heater for polycaprolactone is kept at 110° C. while the nozzle heater is adjusted to 90° C.

### Scaffold Surface Modification

Three different modification techniques were employed to modify the scaffold surface: 1) oxygen-based plasma modification, 2) protein coating, and 3) plasma/protein coating combination modification.

For plasma modification, the plasma system (PDC 32G, Harrick Scientific Inc., New York) included a radiofrequency generator capable of 0-18 W at a frequency range of 8-12 MHz, a vacuum pump, a helical internal electrode around the reactor, and instrumentation for pressures. 3D polycaprolactone scaffolds were placed inside the chamber and exposed to the plasma for 3 minutes at 10 psi with a pure oxygen gas flow rate of 1 standard liter/min and power of 18 W at room temperature.

For protein coating, fibronectin was used. The scaffolds were soaked into different concentration of fibronectin solution prepared from a dilution of 1 mg/mL FN adhesion promoting peptide (Sigma, Cat # F3667) with Phosphate Buffer Saline (PBS). Next, the scaffolds were stored in refrigerator for 12 hours. Following fibronectin coating, the scaffolds were washed with PBS four times. The remaining protein adherence sites were blocked with 2% Bovine Serum Albumin (Sigma, Cat # A2153) to prevent nonspecific interactions of the cells with the polymer substrate and to prevent the adsorption of additional proteins from serum-containing media that could influence cell behavior. BSA was applied to the fibronectin coated scaffolds that were stored for 2 hours at room temperature. Then, the scaffolds were washed with PBS and moved to laminar hood for cell seeding.

For the plasma/protein coating combination, the scaffolds were first modified with plasma and then coated with fibronectin as above.

### Surface Characterization

The assess the degree of hydrophilicity and the solid surface energy, the contact angle measurement of 2 μL of probe liquids (diiodomethane, glycerol, ultra pure water) on surface was measured. The measurements were taken at least four times to obtain an average. All measurements were conducted at room temperature. The measured contact angles (θ) were used to calculate the solid surface energy (σₛ) based on Young's Equation (Volpe et al., 2002, Colloids and Surfaces a-Physicochemical and Engineering Aspects 206:47-67):

σₛ=γₛₗ+σₗ·cos θ (1)

In Young's Equation, the surface energy of probe liquids (σₗ) can be obtained from the literature for different probe liquids, and the solid/liquid interfacial energy (γₛₗ) for polymers can be calculated based on Owens-Wendt method (Wu, 1971, Journal of Polymer Science Part C: Polymer Symposia 34:19-30):

$${γ}_{sl} = {σ}_{s} + {σ}_{l} - ( \frac{4 {σ}_{l}^{D} {σ}_{s}^{D}}{{σ}_{l}^{D} + {σ}_{s}^{D}} + \frac{4 {σ}_{l}^{P} {σ}_{s}^{P}}{{σ}_{l}^{P} + {σ}_{s}^{P}} ) \qquad ( 2 )$$

where superscripts D and P represent the dispersive and polar components, subscripts s and 1 denote solid and liquid phases, respectively. By substituting interfacial tension (γₛₗ) into Young's Equation (Eq.1) and by arranging the equation, it is possible to calculate the polar and disperse fractions of the surface energy with the aid of a single linear regression from the contact angle data of various liquids (Volpe et al., 2002, Colloids and Surfaces a-Physicochemical and Engineering Aspects 206:47-67). FIG. 9 shows an example of such a plot drawn by using six different probe liquids. In the figure, each star is plotted according to the probe liquid's contact angle data on the substrate and liquid surface energy parameters (polar and dispersive) data from the literature.

To assess the surface topography and surface chemistry, atomic force microscopy (AFM) was used to evaluate the surface topography on polycaprolactone in terms of surface features and surface roughness. A Dimension 3100 AFM (Digital Instruments, USA) was used in tapping mode at ambient conditions. The scan size was 5 μm, and the samples were scanned at a frequency of 1 Hz. Nanoscope 5.12 software was used to determine the surface characteristics of a surface quantitatively from AFM image data. To remove the artifacts such as artificial curvatures, tilt and distortion, all images were subjected to a third order flatten using the Nanoscope software. Root-mean-square roughness (RRMS), which is the standard deviation from the mean surface level of the image, was measured by Nanoscope software. In addition, phase AFM images of polycaprolactone film surface over a 5×5 μm square were plotted.

X-Ray photoelectron spectroscopy (XPS, Axis Ultra 165, Kratos-Shimadzu Corporation, USA) was used to identify the changes in near surface compositional depth profiling for modified and unmodified polycaprolactone samples. Spectra was obtained by Kratos Axis Ultra 165 spectrometer using A1 K_{α} (1486.7 eV) beam radiation at a power of 100 W. A take-off angle of 90° with respect to the 1×0.5 mm² sampling area was used. All measurements were taken under vacuum between 10⁻⁹ and 10⁻¹⁰ Torr. Elemental high resolution scans for C₁ₛ, Oₗₛ, Si₂ₛ, Si₂ₚ and N₁ₛ were taken at the pass energy of 20 eV. A value of 285.0 eV for the hydrocarbon C₁ₛ core level was used as the calibration energy for the binding energy scale.

### Cell-Scaffold Interaction

For the cell-scaffold interaction analysis, 7F2 mouse osteoblast cells (CRL-12557, American Type Culture Collection, Rockville, Md.) were used. The cells were cultured in alpha-minimum essential medium (α-MEM) (Gibco, Grand Island, N.Y.) containing 10% fetal bovine serum (Hyclone, Logan, Utah), 2 mM L-glutamine and 1 mM sodium pyruvate without ribonucleosides and deoxyribonucleosides. Beginning on the third day of the culture, the medium was supplemented with 10 mM β-glycerophophate (Sigma,) and 50 μg/mL ascorbic acid (sigma,) to promote the osteoblastic phenotype. When the cells reached confluency, the 7F2 cells were trypsinized and resuspended in medium at a concentration of 1×10⁶ cells/mL. The osteoblast cells were seeded onto scaffolds by adding a 1 mL droplet of cell suspension to the top of each scaffold. After 2 hours of incubation, the scaffolds were moved to new wells to leave behind any unattached cells. Then, 1 mL fresh osteogenic medium was added to each individual well. The cell-scaffold construct was maintained in an incubator (37° C. and 5% CO₂).

### Measurement of Cell Adhesion

The changes in the degree of cell adhesion strength after surface modifications were measured by shear flow assay. In shear flow assay apparatus, the upper plate was a flat glass coverslip while the lower plate was the place in where the flow chamber was located. The treated surface incubated with cells was first placed inside the flow chamber (25×75×1 mm) (W×L×H) and laminar flow (Re<2) was initiated between the plates. It is assumed that the shear stress at the wall (τ_{w}) is equal to detachment shear stress at the surface of the cells.

τ_{w}=6 μQ/wh² (3)

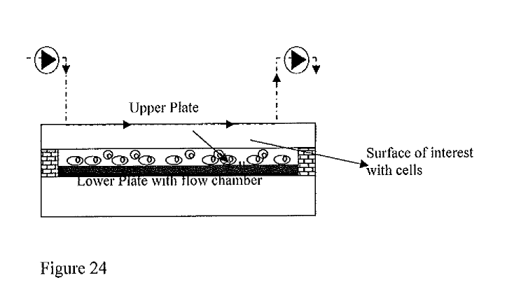

In FIG. 24, the schematic view of the shear flow assay apparatus is shown. Before applying the flow, a cell suspension with 1.0×10⁵ cells was seeded onto the treated surface (n=3) and the cells were permitted to settle and attach for two hours at incubator condition. After the incubation, the sample was placed inside the flow chamber and the medium was initiated between the plates with 675 ml/min for 1 min, then the attached cells on the treated surface were counted by alamarBlue™.

### Measurement of Cell Proliferation and Differentiation

To assess metabolic activity, the non-toxic alamarBlue™ (aB) assay (Biosource International, USA) was performed to measure the cell proliferation on unmodified and modified polycaprolactone scaffolds (n=4 for each group and each measurement day) at 0, 3, 7, 14, and 21 days after seeding. At the day of characterization, 10% (v/v) of aB assay solution was added to each well and allowed to incubate for 4 hours at 37° C. and 5% CO₂ condition. After incubation, 800 μL solution of color product was taken out of each well and put into a 24 well-plate to measure the fluorescence intensity by microplate fluorometer (Genius, TECAN, USA) at 535 nm excitation and 560 nm emission wavelengths.

To quantify alkaline phosphatase (ALP activity), mouse osteoblast cells attached to polycaprolactone scaffolds (n=4 for each group and each measurement day) were assayed with p-nitrophenylphosphate (pNPP) (Sigma, Cat# N7653) at 7, 14, and 21 days after seeding. On the of day measurement, the scaffolds were removed from the medium and washed with PBS twice. Then they were submerged into 1 mL of 1% Triton x100 solution for an hour. During this one hour period, the solution was agitated several times for the cell lysis. After one hour, 0.5 mL solution was incubated with 0.5 mL pNPP for 45 min at 37° C. The production of p-nitrophenol in the presence of ALP was monitored with microplate fluorometer by the absorbance of 405 nm wavelength. The ALP activity was expressed as unit per cell number.

To quantify osteocalcin release, the amount of osteocalcin synthesized by mouse osteoblast cells was measured by an enzyme-linked immunosorbent assay (ELISA) kit (Biomedical Technologies Inc., Stoughton, Mass.) at 7, 14, and 21 days after seeding for each group (n=4). The protocol supplied from manufacturer was followed during the sample preparation for measurement. The absorbance value the prepared solution was measured at 450 nm by microplate fluorometer (Genius, TECAN, USA). The resulting absorbance values for the samples were converted to osteocalcin concentration (pg/mL) using a standard curve generated from known concentrations of osteocalcin standard solutions.

### Statistics

The statistical significance was determined by analysis of variance (ANOVA) and Tukey post-hoc test at the significance level of less than 0.05 (P<0.05) using SPSS® version 14 for Windows® software package.

The Results of Example 3 are now described.

### Effect of Surface Modification on Surface Hydrophilicity and Energy

The results from contact angle measurements on unmodified, plasma modified, protein coated and the plasma/protein modified polycaprolactone samples are given in Table 4. The measurements were conducted immediately after modification techniques for the three probe liquids ultrapure water, diiodomethane, and glycerol. The±represents the standard deviation with n=4 for each modification technique and for each probe liquids.

TABLE 4
Measured static contact angle data probe liquids on unmodified,
protein coated, plasma modified, and the plasma/protein modified
polycaprolactone surface.

| Modification | Static Contact Angle (°) | | |
|---|---|---|---|
| Type | Ultra Pure Water | Glycerol | Diiodomethane |
| Unmodified | 72 ± 2 | 69 ± 3 | 27 ± 1 |
| Protein | 31 ± 3 | 52 ± 5 | 28 ± 3 |
| Plasma | 29 ± 1 | 32 ± 2 | 21 ± 2 |
| Plasma/Protein | 24 ± 1 | 32 ± 4 | 24 ± 3 |

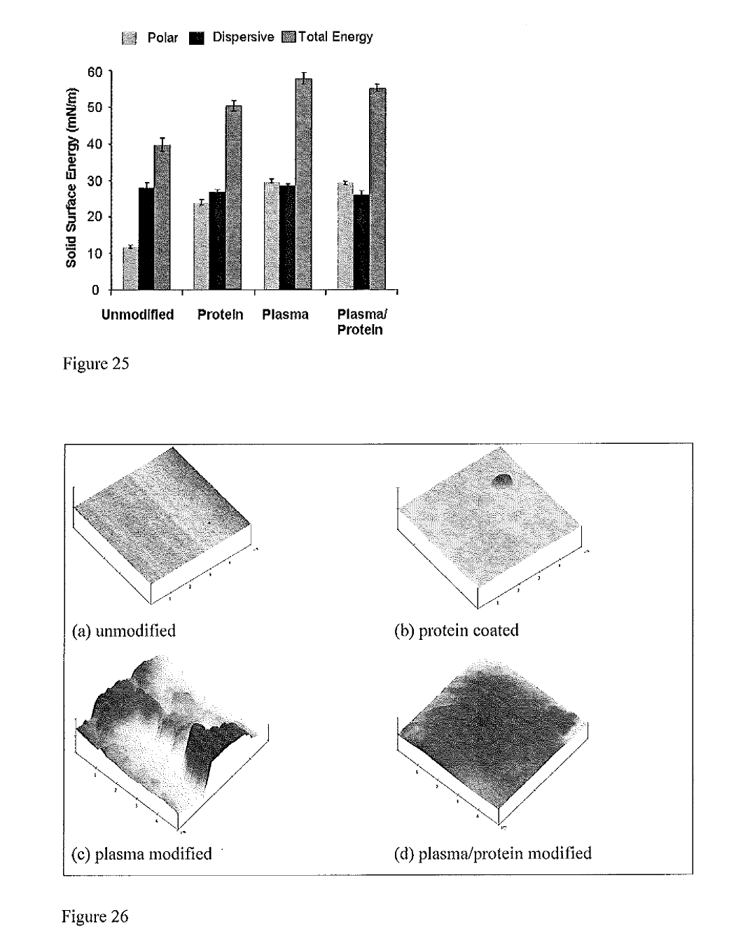

The measured contact angles were used as the basis for the calculation of solid surface energies. The total surface energy (σₛ) of polycaprolactone and its polar (σₛ^{P}) and dispersive (σₛ^{D}) components before and after oxygen plasma modification, were calculated by using Owens-Wendt's method (Brant et al., 2002, Journal of Membrane Science 203:257-273; Gindl et al., 2001, Colloids and Surfaces a-Physicochemical and Engineering Aspects 181:279-287; Lopes et al., 1999, Journal of Biomedical Materials Research 45:370-375). As an input in this method, the measured contact angle data (Table 4) and liquid surface tension components (Yildirim et al., 2008, Plasma Processes and Polymers 5:397-397) of three probe liquids were used. FIG. 25 shows the variation in the total, polar and dispersive solid surface energy of polycaprolactone with plasma, protein and plasma/protein modification techniques.

The increment in total solid surface energy on protein coated, plasma modified and plasma/protein (combined) modified polycaprolactone surfaces were significantly higher compared to the unmodified polycaprolactone (FIG. 25). Among them, plasma modification showed the highest the total solid surface energy, while plasma/protein (combined) modified and protein coated followed it, respectively. Since the total surface energy is the summation of dispersive and polar component of the surface energy, it can be said that the increments in total energy come from the polar component in all four groups of polycaprolactone. The dispersive component did not have any contribution in the total energy increment because it remained essentially constant with all three modification techniques.

### Effect of Surface Modification on Surface Topography

The changes in polycaprolactone surface structure and roughness after modifications were measured and imaged by Atomic Force Microscopy (AFM). The root mean square roughness (RMSR) for unmodified, protein coated, plasma modified, and the plasma/protein modified polycaprolactone are given in Table 5. In Table 5, the ±represents the standard deviation of the roughness for three samples (n=3) for each modification techniques. The AFM phase images (the three-dimensional) for unmodified, protein coated, plasma modified, and the plasma/protein modified polycaprolactone are given in FIG. 26.

TABLE 5
Surface root mean square roughness (RMSR) for unmodified and
modified polycaprolactone

| | | | Plasma | Plasma/Protein |
|---|---|---|---|---|
| | Unmodified | Protein Coated | Modified | Modified |
| RMSR (nm) | 41 ± 8 | 56 ± 4 | 150 ± 12 | 78 ± 9 |

Based on roughness measurements and phase images it was observed that the unmodified polycaprolactone surface was relatively smooth with a roughness of 41±8 nm (FIG. 26a and Table 5). After coating with fibronectin, the individual features on the unmodified polycaprolactone surface, the surface roughness was 56±4 nm (FIG. 26b). In contrast, on polycaprolactone surfaces exposed to plasma modification, obvious granular structures with large peaks and valleys were formed with an increase in roughness to 150±12 nm (Table 5 and FIG. 26c). The roughness of plasma/protein (combined) modified polycaprolactone surface is 78±9 nm with a relatively uniform surface features (Table 5 and FIG. 26d).

### Effect of Surface Modification on Surface Chemistry

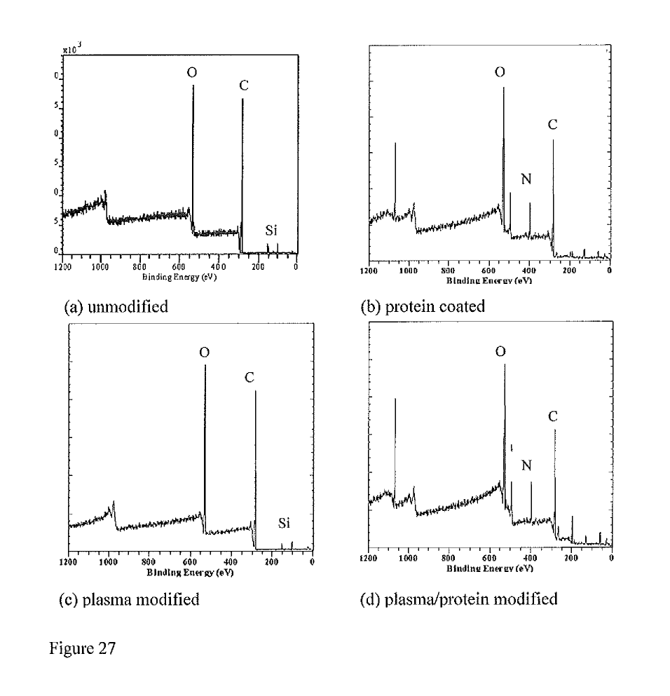

X-ray Photoelectron Spectroscopy (XPS) analysis of modified polycaprolactone samples was conducted to investigate both the changes in surface chemical composition and the types of functional groups introduced by modification techniques. Table 6 shows the surface atomic concentration of unmodified, protein coated, plasma modified and plasma/protein modified polycaprolactone samples. FIG. 27 shows the survey XPS spectra of unmodified and modified polycaprolactone samples.

TABLE 6
Surface atomic concentration for unmodified and modified
polycaprolactone

| | Atomic | | |
|---|---|---|---|
| Modification | Concentration (%) | | |
| Type | O | C | N |
| Unmodified | 22.13 | 72.00 | — |
| Protein | 21.64 | 64.65 | 7.09 |
| Plasma | 23.95 | 73.59 | — |
| Plasma/Protein | 20.98 | 59.33 | 7.93 |

The survey XPS spectra indicate that the atomic concentration of the polycaprolactone surface does change with the modifications. The surface atomic concentration of protein coated and plasma/protein combined modified samples are quite different than the counterparts modified with only plasma. The protein immobilization can be distinguished from nitrogen features peaking at 396.8 eV in XPS spectra (FIG. 27b and FIG. 27d). Furthermore, the increase in nitrogen atomic concentration at the surfaces of protein coated and plasma/protein combined modified samples is given in Table 7. Besides the change in atomic concentrations, few contaminations by silicon, sodium, chloride and potassium were observed on polycaprolactone surface. Possible reasons for the contamination included the sample preparation procedure and/or the pollutant present in the plasma chamber.

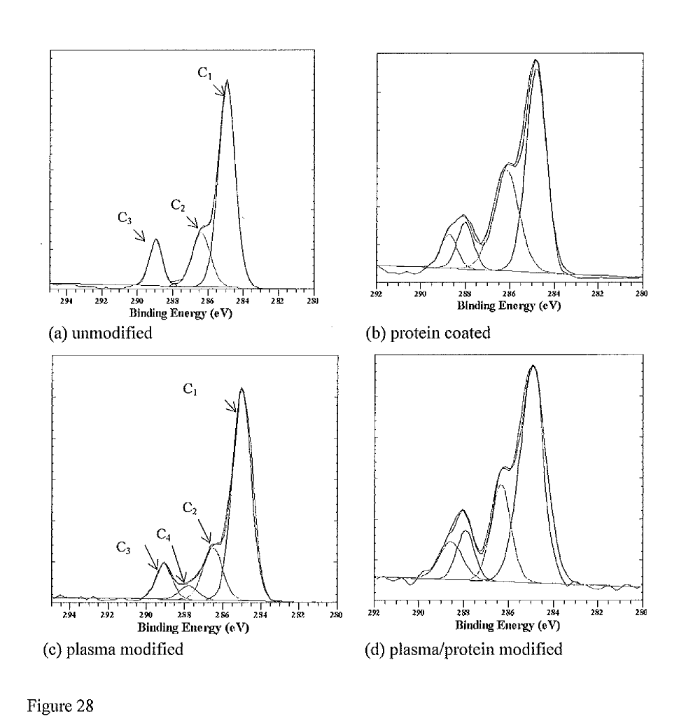

In addition, since oxygen was the element besides carbon present at significant concentrations (Table 6), the authors investigated the chemical state of oxygen at the surface. In order to identify the changes in fraction of various functional groups on modified polycaprolactone samples, high resolution scans for C₁ₛ, O₁ₛ, Si₂ₛ, Si₂ₚ and N₁ₛ were taken. FIG. 28 shows the deconvoluted C₁ₛ peak survey XPS spectra of unmodified and modified polycaprolactone samples. The C₁ₛ spectra were deconvulated into four peaks which are for graphitic (C—C, C—H) at 285 eV, hydroxyl (C—OH, C—O) at 286.4 eV, carboxyl (O—C═O, COOH) at 288.9 eV and carbonyl group (C═O) at 287.8 eV. The fraction of graphitic, hydroxyl, carboxyl and carbonyl functional groups on unmodified, protein coated, plasma modified and plasma/protein modified polycaprolactone samples were shown in Table 7.

TABLE 7
The fraction of functional groups on polycaprolactone surface

| | Graphitic | Hydroxyl | Carboxyl | |
|---|---|---|---|---|
| | C—C | C—OH | —O—C═O | Carbonyl |
| | C—H | C—O | —COOH | C═O |
| Modification | 285 eV | 286.4 eV | 288.9 eV | 287.8 eV |
| Type | C1 (%) | C2 (%) | C3 (%) | C4 (%) |
| Unmodified | 68.93 | 19.53 | 11.54 | — |
| Plasma | 67.58 | 17.81 | 10.24 | 4.37 |
| Protein | 51.34 | 32.54 | 6.87 | 9.25 |
| Plasma/Protein | 60.53 | 20.56 | 9.59 | 9.32 |

From FIG. 28 and Table 7, it can be observed that due to the chemical composition of polycaprolactone ((CH2)₅—C═O—O)), even for the unmodified sample we can observe the fraction of hydroxyl (C₂ₛ) functional groups shouldering at 286.4 eV, and carboxyl (C₃ₛ) functional groups shouldering at 288.9 eV on the surface (FIG. 28a). For unmodified samples, the functional groups and their fractions on the surface are as follows; graphitic (68.93%), hydroxyl (19.53%) and carboxyl groups (68.93%) (Table 7).

Through modifications with plasma, protein, and combined technique the fraction of graphitic group (C₁ₛ) decreased, while additional carbonyl (C═O) functional group was introduced on polycaprolactone surface (Table 7 and FIG. 28). From FIG. 28a, it can be observed that the unmodified sample showed no shoulder at 287.8 eV (C₄ₛ) representing carbonyl groups on the surface. However with the modifications we can observe the C₄ₛ shouldering at XPS spectra given at FIGS. 28b and 28c and 28d. The FIG. 28 and Table 7 also indicate that plasma, protein and combined modification enriched the total oxygen containing functional groups content of the surface. The most of the oxygen containing functional group on the surface found as hydroxyl (C2s) functionalities, but a significant amount of carboxyl (C3s) and carbonyl (C4s) functionalities were also detected at all modified samples (Table 7).

### Effect of Surface Modification on Cellular Function

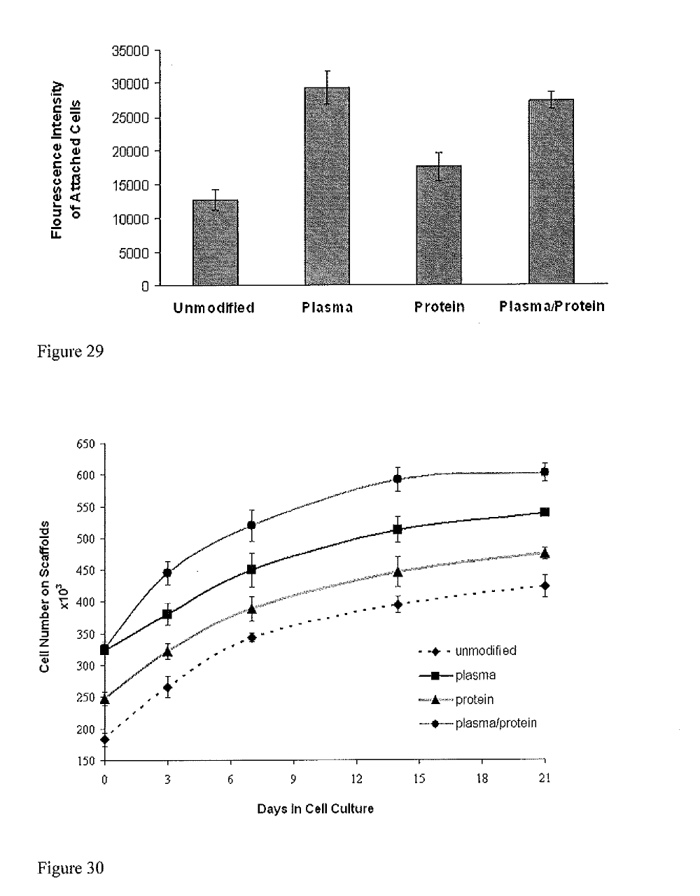

A cell adhesion assay was used to assess cell attachment. Cells in different sample groups were exposed to the shear stress in a parallel-plate flow chamber. The results of the cell attachment were presented in FIG. 29. After the exposure to shear flow, the number of cells on unmodified and protein coated polycaprolactone were significantly lower compared with the numbers on plasma and combine modified polycaprolactone. Both groups exposed to plasma (plasma modified and combined) had almost twice the amount of cells compared to unmodified and protein coated scaffolds under the same flow. However, the cells on unmodified and protein coated samples showed statistically no difference among each other. Same conclusion can be done for the cell number on plasma modified and combined modified polycaprolactone.

The proliferation of cells associated with the scaffolds was assessed using a 21-Day in vitro cell culture assay. FIG. 30 shows the number of cells associated with the unmodified, plasma-modified, protein-coated and combined (plasma+protein) modified scaffolds. Twenty-one-day in vitro cell culture showed that throughout the culture period cells on combined modified scaffolds showed higher rate of proliferation compared to the counterparts on unmodified, plasma modified and plasma coated scaffolds. Different than the rest of characterization day, at Day 0, the cells on plasma modified and combined modified showed higher metabolic activity than protein coated and unmodified ones without showing significant difference among themselves. At Day 3, the numbers of cells on combined modified scaffolds was significantly higher than those on plasma modified, protein coated and unmodified scaffolds. This trend was continued throughout the 21 days of study. However, starting at Day 7, the cells number were leveling off which was the indication of starting point of early differentiation phase.

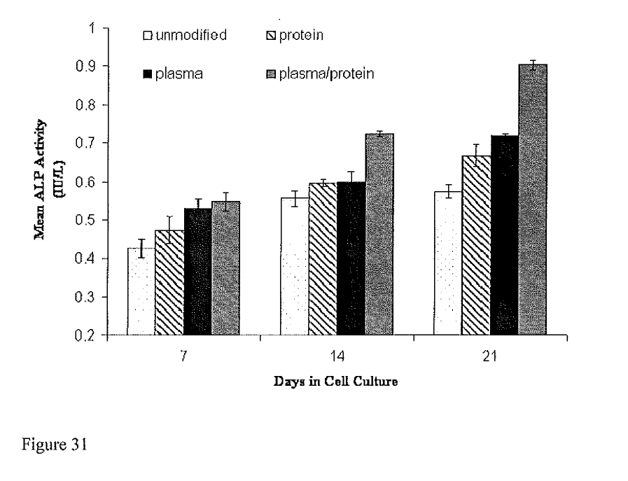

Differentiation of cells was assessed using an alkaline phosphatase activity assay. Alkaline phosphatase activity (ALP) is the one of the earliest marker of osteoblast differentiation and its expression persists throughout the maturation of osteoblast (Holtorf et al., 2005, Biomaterials 26:6208-6216). The ALP activities of cells associated with unmodified and modified polycaprolactone scaffolds are shown in FIG. 31 as unit per cell number. Following Day 7, ALP activities were up-regulated in all groups indicating the starting of differentiation. At Day 14, some distinction started to be observed in ALP activities with those scaffolds modified with plasma and plasma/protein (combined). Among those, plasma/protein modified scaffolds had greater ALP activity than only plasma modified counterpart with a significant difference. By day 21, there was an increase in ALP activity for plasma-modified and protein-coated scaffolds, but the difference was not significant. At Day 21, ALP activity on combined (plasma+protein) modified scaffolds had the highest value compared to the other groups.

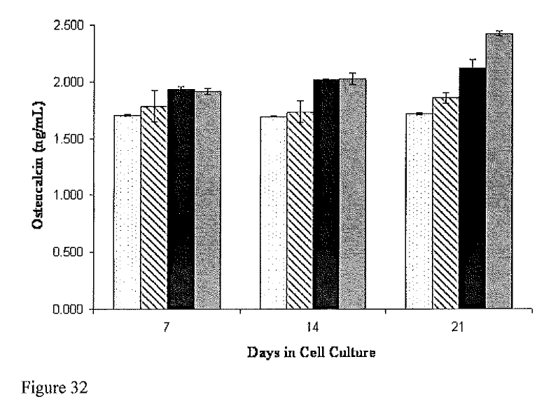

To confirm the analysis of ALP activity, the osteocalcin level in the cell culture medium was measured. Expression of osteocalcin is the most specific marker for osteoblast, and its expression has been onset of mineralization phase. FIG. 32 shows the amount of osteocalcin protein secreted by cells cultured on unmodified and modified polycaprolactone scaffolds for 21 days of in vitro culture. The data in FIG. 32 suggest that osteocalcin was continuously expressed from Day 7 to Day 21 on unmodified polycaprolactone scaffolds at similar levels. There was a slight increase when cultured on protein-coated, scaffold on day 21 when compared to the unmodified scaffold. From Day 7 to Day 14, there was no statistical difference in osteocalcin level between unmodified and protein-coated scaffold. In contrast, osteocalcin was strongly secreted when cultured on plasma modified and combined modified polycaprolactone scaffolds. Particularly after Day 7, the difference in secreted osteocalcin level was distinguishable on plasma-modified and combined-modified scaffolds. In accordance with ALP data, by Day 21, a statistically significant difference was observed between all four modified groups with the following order; plasma/protein>plasma>protein>unmodified.

### Example 4

### Functional Freeform Microplasma Surface Patterning

The microplasma system and method is operated at a non-thermal and atmospheric pressure environment to conduct a maskless, stampless functionalized surface treatment. In some embodiments, the system and methods further include the printing of cells and/or biomolecules onto the functionalized substrate surface.

The microplasma system and method uses a maskless, stampless surface patterning process to create spatially defined physical, topological and chemical features on a biopolymer substrate surface. Such a functionalized patterned substrate surface can be used, for example, to guide the organization of the cells and biomolecules.

The microplasma system and method generates micron-scale functionalized patterns on a substrate without using any chemical, solvent, mask or stamp. The substrate is functionalized using dielectric barrier discharge (DBD) technique. DBDs are non-equilibrium plasmas which are easy to operate at atmospheric pressure (Ayan et al., 2009, Journal of Physics D-Applied Physics 42). A pulsed power supply with variable frequency is employed to generate the plasma. The plasma system consists of a high voltage electrode inserted coaxially in a dielectric (borosilicate glass or quartz) tube and ground electrode wrapped around the tube from outside. The gas or gas mixture is purged through an annular gap between coaxial electrode and dielectric tube. When the high voltage electrode is powered, plasma ignites between the electrodes and a micro-scale plasma jet appears at the tip of the nozzle. FIG. 33 represents the schematic view of the microplasma system and its components. Once the microplasma contacts with the surface of biopolymer, we expect to change the topography and chemistry on the plasma exposed area. Depending on the microplasma operation parameters, such as plasma power, gas flow rate, gas composition, and nozzle tip diameter, we expect to have certain control on chemical composition and topological features of polymer surface. The size of the topographic features created by microplasma will be measured by atomic force microscopy (AFM) and scanning electron microscopy (SEM). The amount and distribution of introduced functional groups on the microplasma functionalize biopolymer surface will be characterized by x-ray photoelectron microscopy (XPS).

### Example 5

### Integration of Microplasma System with Bioprinting System

The methods and materials of Example 5 are now described.

The microplasma system and methods described in Example 4 is integrated with a freeform fabrication based bioprinting system to perform both the freeform generation of microplasma surface patterning and the printing of cells and biomolecules. One example of such a bioprinting system is described in U.S. patent application Ser. No. 10/540,968 and is incorporated by reference herein in its entirety (See also Chang et al., 2008, Tissue

Engineering Part C-Methods 14:157-166).

A schematic of an embodiment of such an integrated system is shown in FIG. 34. As depicted in FIG. 34, the microplasma functionalizes the biopolymer surface based on designed pattern and the bioprinting system prints cells or biomolecules on the functionalized patterned surfaces. In various embodiments of the system and methods of the invention, the microplasma functionalization of the biopolymer occurs before the bioprinting step. In other embodiments, the bioprinting step occurs concurrently with the microplasma functionalization step.

The system allows the plasma surface functionalization and direct cell/biomolecule printing to be accomplished within one system through concurrent or sequential processes. The system enables functionalization of the biopolymer surface with designed patterns and print the cells subsequently without requiring the preparation and use of a mask, mask design and mask manufacturing.

### Effect of Maskless Microplasma Generated Cell Patterning on Cellular Function,

The effect of maskless microplasma generated cell patterning on cellular functions, including the attachment, proliferation and differentiation, metabolic activity and differentiation is examined as elsewhere described herein. Other assays are conducted as elsewhere described herein, including the MTT assay and assessments of ALP activity.

### Development of a Biomolecule Printing System

A biomolecule printing system is developed which is capable of printing different types of biomolecules according to the pre-design model (See Chang et al., 2008, Tissue Engineering Part C-Methods 14:157-166). By contrast to other freeform fabrication systems, this biomolecules printing system allows the concurrent printing of different types of biomolecules in controlled amounts with precise spatial positioning. The biomolecules printing system is consists of two sub-systems: the data processing system and the 3D motion system. The data processing system processes CAD model or pattern and converts it into a layered process toolpath. The 3D motion system consists of X-Y-Z axis that are actuated by one AC servo motors driven by servo drivers. The motion of the axis can be controlled by an in-house developed computer program. The biomolecules printing nozzles are assembled to the 3D motion system that can move the axis in 20×20×20 cm³ space. Nozzles can be in different sizes and containing different types of biomolecules. Each nozzle has its own operation parameters so we have a chance to adjust the nozzles parameters as requires such as the printed biomolecule amount, nozzle moving speed, and activation or deactivation of different nozzles at the same time.

The Results of Example 5 are now described.

### Microplasma Generated Surface Functionalization

Atomic force microscopy (AFM) was used to observe nano-features and their distribution on biopolymer before and after the microplasma functionalization. AFM has been commonly used on plasma treated surfaces due to the ease of sample preparation and excellent resolution (Yildirim et al., 2008, Plasma Processes and Polymers 5:58-66). A Dimension 3100 AFM (Digital Instruemnts, USA) was used in tapping mode at ambient conditions. The changes in surface roughness with microplasma functionalization time is given in Table 8.

TABLE 8
Surface statistical parameters of polycaprolactone films for untreated
and treated samples

| Plasma | | | |
|---|---|---|---|
| Functionalization Time | RMS (nm) | Rₐ (nm) | Rₘₐₓ (nm) |
| Untreated | 18.025 | 13.116 | 175.16 |
| 1-min | 26.055 | 19.798 | 214.55 |
| 3-min | 33.823 | 25.619 | 214.37 |
| 5-min | 50.340 | 38.932 | 318.96 |

The root-mean-square roughness (RRMS), which is the standart deviation from the mean surface level of the image, also the maximal height diffence, Rₘₐₓ, and the average roughness Rₐ were measured by Nanocope software and listed in Table 8. While for unmodified polymer the average roughness (Rₐ) is 13.116 nm, this value was increased with the prolonged plasma functionalization time. For the 5-min plasma functionalization, the average roughness value was trippled to 38.932 nm comparing to unmodified substrate.

### Introduction of Functional Groups on Biomaterial Surface

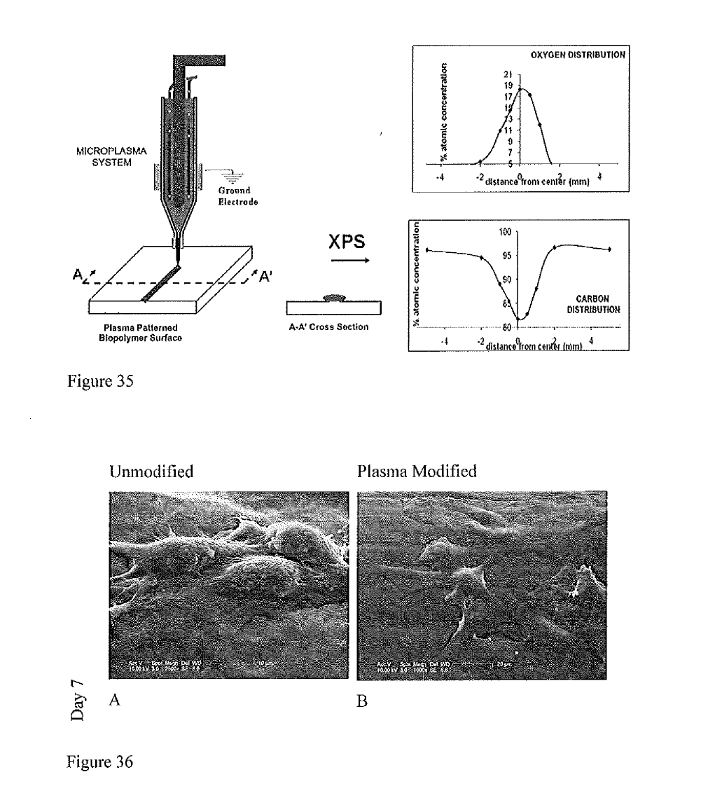

The effect of microplasma functionalization of polycaprolactone surface was examined. The changes in polycaprolactone surface chemistry after oxygen-based plasma functionalization were determined by X-ray Photoelectron Spectroscopy (XPS). Spectra was obtained by Kratos Axis Ultra 165 spectrometer using Al K_{α} (1486.7 eV) beam radiation at a power of 100 W. A value of 285.0 eV for the hydrocarbon C₁ₛ, core level was used as the calibration energy for the binding energy scale. Specifically, to observe the chemical changes on the patterned surface, the surface was modified in a pattern and the chemical composition at the cross section of the pattern areas was examined as described in FIG. 35.

In FIG. 35, the XPS results suggest that surface atomic concentration of biopolymer is changed after plasma functionalization. The fraction of carbon containing groups decreased with the plasma exposure, while the concentration of oxygen containing functional groups increased. The highest amount of oxygen (or lowest amount of carbon) concentration was observed on the center location of the patterned line and this concentration started to decrease when the distance from the center increased, suggesting that the change in surface chemistry is more dominant on the area where the microplasma passed through. The increased distance in longitudinal direction from the center of pattern might be caused by the glow or after effect of the plasma.

### Effect of Microplasma Surface Functionalization On Cell Morphology

The effect of microplasma surface modification on cell morphology was assessed by culturing mouse osteoblast cells on microplasma modified and unmodified polycaprolactone samples. The changes in cells' morphology were assessed by scanning electron microscopy (SEM) after seven days in culture. On characterization day, the cell-biomaterial constructs were removed from the medium before fixing the cells with 3% gluteraldehyde (Sigma-Aldrich, USA) for 2.5 hours at 4° C. After fixation, the scaffolds were washed with PBS and gradually dehydrated through 50%, 70%, 80%, 90%, and 100% ethanol solutions for 30 min each. Following the dehydration, the samples were kept in 4° C. for overnight and next day sputter coated with Plt layer to look under SEM (FEI/Phillips XL30). The cell morphologies on microplasma modified and unmodified samples are given in FIG. 36.

FIG. 36 suggests that cells on plasma modified samples (FIG. 36B) exhibited elongated morphology on biopolymer surface with high degree of spreading. In contrast, on unmodified surface osteoblast cells were hardly attached and preserved their round shape (FIG. 36A), suggesting that plasma modification improve the cell spreading over the polymer surface.The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A microfluidic system for monitoring or detecting a change in a characteristic of an input substance, the microfluidic system comprising:
a cover platform having an inlet for delivery of an input substance and an outlet for removal of an output substance;
a substrate platform having a tissue chamber in a substrate body of the substrate platform and a three-dimensional tissue analog comprising cells mixed with a basement membrane matrix (BME);
a first microfluidic channel in fluid communication with the inlet for delivery of the input substance and the tissue chamber;
a second microfluidic channel in fluid communication with the outlet for removal of the output substance, provided that the substrate platform and the cover platform are superimposed to form a sealed assembly;
an input substance unit; and
an optional pumping assembly and detecting unit.

2. The microfluidic system of claim 1, wherein the substrate platform comprises the first microfluidic channel and the second microfluidic channel in fluid communication with the tissue chamber.

3. The microfluidic system of claim 1, wherein the cover platform comprises the first microfluidic channel and the second microfluidic channel in fluid communication with the tissue chamber.

4. The microfluidic system of claim 1, wherein at least one of the cover platform and the substrate platform comprises a surface with an improved hydrophilicity.

5. The microfluidic system of claim 1, wherein at least one of the cover platform and the substrate platform are made of a polymer, glass, a ceramic, a metal, an alloy, or a combination thereof.

6. The microfluidic system of claim 1, wherein the cover platform is made of a plasma treated glass and the substrate platform is made of a plasma treated biologically-compatible polymer composed of a plurality of siloxane units.

7. The microfluidic system of claim 1, wherein the tissue analog is at least one selected from the group consisting of heart, stomach, kidney, intestine, lung, liver, fat, bone, cartilage, skeletal muscle, smooth muscle, cardiac muscle, bone marrow, muscle, brain, and pancreas.

8. The microfluidic system of claim 1, comprising a plurality of microfluidic channels.

9. The microfluidic system of claim 1, comprising a plurality of tissue chambers.

10. A method of monitoring or detecting a change in a characteristic of an input substance, the method comprising:
providing the microfluidic system of claim 1;
providing the input substance unit comprising the input substance;
directing the input substance into the microfluidic system, wherein the input substance flows through the inlet for delivery of the input substance and the first microfluidic channel into the tissue chamber having the tissue analog;
removing the output substance from the microfluidic system via the second microfluidic channel and the outlet for removal of the output substance;
obtaining at least a portion of the input substance prior to entry into the microfluidic system and at least a portion of the output substance after exiting the microfluidic system;
measuring the characteristic of the input substance prior to entry into the microfluidic system and measuring the characteristic of the output substance after exiting the microfluidic system; and
comparing the measured characteristic of the input substance prior to entry into the microfluidic system with the measured characteristic of the output substance after exiting the microfluidic system;
thereby monitoring or detecting a change in the characteristic of the input substance.

11. The method of claim 10, wherein the input comprises a drug.

12. The method of claim 11, wherein said monitoring or detecting the change in the characteristic of the input substance comprises:
collecting the output comprising a metabolite having a detectable characteristic;
detecting the detectable characteristic; and
correlating the detectable characteristic to at least the extent and rate of metabolism of the input substance.

13. A microplasma system for functionalized patterning of a tissue engineering substrate, the system comprising a microplasma nozzle fixed adjacent to a substrate material that is affixed to a platform moveable by a motion control system to position and move the platform in the X, Y and Z directions in relation to the fixed microplasma nozzle to create a functionalized pattern on the surface of the substrate material.

14. The system of claim 13, wherein the substrate material is polycaprolactone.

15. A microplasma system for functionalized patterning of a tissue engineering substrate, the system comprising a moveable microplasm nozzle affixed to motion control system to position and move the microplasma nozzle in the X, Y and Z directions in relation to a substrate material to create a functionalized pattern on the surface of the substrate material.

16. The microplasm system of claim 14, wherein said microplasm nozzle is affixed to a multi-nozzle bioprinting system comprising:
a data processing system that processes a designed scaffold model and converts it into a layered process tool path;
a motion control system driven by the layered process tool path; and
a material delivery system comprising multiple nozzles of different types;
wherein at least one of the nozzles deposits at least one substrate material, and at least one of the nozzles deposits at least one type of cell, and at least one of the nozzles deposits at least one biomolecule;
thereby constructing a scaffold having a microplasma functionalized pattern.

17. The system of claim 15, wherein the substrate material is polycaprolactone.

18. A method of creating a functionalized pattern on the surface of a tissue engineering substrate comprising the steps of:
fixing a microplasma nozzle adjacent to a substrate material that is affixed to a platform moveable by a motion control system; and
moving the platform in the X, Y and Z directions in relation to the fixed microplasma nozzle to create a functionalized pattern on the surface of the substrate material.

19. The method of claim 18, wherein the substrate material is polycaprolactone.

20. A method of creating a functionalized pattern on the surface of a tissue engineering substrate comprising the step of moving a microplasm nozzle affixed to motion control system in the X, Y and Z directions in relation to a substrate material to create a functionalized pattern on the surface of the substrate material.

21. The method of claim 20, wherein the microplasma nozzle is integrated into a multi-nozzle bioprinting system.

22. The method of claim 20, wherein the substrate material is polycaprolactone.
